SEQUENCE LISTING (6 sequences)

SEQ ID NO: 1 - protein, 848 aa, Homo sapiens
MNSSSANITYASRKRRKPVQKTVKPIPAEGIKSNPSKRHRDRLNTELDRLASLLPFPQDVINKLDKLSVLRLSVSYLRAKSFFDVALKSSPTERNGGQDNCRAANFREGLNLQEGEFLLQALNGFVLVVTTDALVFYASSTIQDYLGFQQSDVIHQSVYELIHTEDRAEFQRQLHWALNPSQCTESGQGIEEATGLPQTVVCYNPDQIPPENSPLMERCFICRLRCLLDNSSGFLAMNFQGKLKYLHGQKKKGKDGSILPPQLALFAIATPLQPPSILEIRTKNFIFRTKHKLDFTPIGCDAKGRIVLGYTEAELCTRGSGYQFIHAADMLYCAESHIRMIKTGESGMIVFRLLTKNNRWTWVQSNARLLYKNGRPDYIIVTQRPLTDEEGTEHLRKRNTKLPFMFTTGEAVLYEATNPFPAIMDPLPLRTKNGTSGKDSATTSTLSKDSLNPSSLLAAMMQQDESIYLYPASSTSSTAPFENNFFNESMNECRNWQDNTAPMGNDTILKHEQIDQPQDVNSFAGGHPGLFQDSKNSDLYSIMKNLGIDFEDIRHMQNEKFFRNDFSGEVDFRDIDLTDEILTYVQDSLSKSPFIPSDYQQQQSLALNSSCMVQEHLHLEQQQQHHQKQVVVEPQQQLCQKMKHMQVNGMFENWNSNQFVPFNCPQQDPQQYNVFTDLHGISQEFPYKSEMDSMPYTQNFISCNQPVLPQHSKCTELDYPMGSFEPSPYPTTSSLEDFVTCLQLPENQKHGLNPQSAIITPQTCYAGAVSMYQCQPEPQHTHVGQMQYNPVLPGQQAFLNKFQNGVLNETYPAELNNINNTQTTTHLQPLHHPSEARPFPDLTSSGFL

SEQ ID NO: 2 - protein, 4 aa, Artificial Sequence
  Description of Artificial Sequence Synthetic peptide
GSHI

SEQ ID NO: 3 - protein, 12 aa, Artificial Sequence
  Description of Artificial Sequence Synthetic peptide
VGIGGGGGGGGG

SEQ ID NO: 4 - protein, 5 aa, Artificial Sequence
  Description of Artificial Sequence Synthetic peptide
GGNGG

SEQ ID NO: 5 - protein, 4 aa, Artificial Sequence
  Description of Artificial Sequence Synthetic peptide
GGNG

SEQ ID NO: 6 - protein, 5 aa, Artificial Sequence
  Description of Artificial Sequence Synthetic peptide
GGQGG

(19) United States
(12) Patent Application Publication
(10) Pub. No.: US 2019/0225683 A1
(43) Pub. Date: Jul. 25, 2019

(54) TARGETING OXAZOLE STRUCTURES FOR THERAPY AGAINST INFLAMMATORY DISEASES

(71) Applicant: THE BRIGHAM AND WOMEN'S HOSPITAL, INC., Boston, MA (US)

(72) Inventors: Richard S. BLUMBERG, Waltham, MA (US); Shankar S. IYER, Boston, MA (US); Thomas GENSOLLEN, Boston, MA (US); Amit GANDHI, Billerica, MA (US)

(73) Assignee: THE BRIGHAM AND WOMEN'S HOSPITAL, INC., Boston, MA (US)

(21) Appl. No.: 15/775,534

(22) PCT Filed: Nov. 14, 2016
(86) PCT No.: PCT/US16/61799

§ 371 Date: May 11, 2018

Related U.S. Application Data
(60) Provisional application No. 62/254,947, filed on Nov. 13, 2015.

(51) Int. Cl. C07K 16/28; G01N 33/53

(52) U.S. Cl. CPC C07K 16/28; G01N 2800/067; C07K 2317/76; G01N 33/5308

(57) ABSTRACT

Described herein are novel compositions, targeted therapeutic methods, and assays for neutralizing and/or inhibiting the activity of “oxazole-containing (OxC) compounds,” to prevent or delay the onset of epithelial barrier dysfunction and chronic inflammation associated with various disorders, such as colitis.

### CROSS REFERENCE TO RELATED APPLICATIONS

This application is a 371 National Phase Entry of International Patent Application No. PCT/US2016/061799 filed on Nov. 14, 2016, which claims benefit under 35 U.S.C. § 119(e) of U.S. Provisional Application Ser. No. 62/254,947 filed on Nov. 13, 2015, the contents of which are herein incorporated by reference in their entirety,

### GOVERNMENT RIGHTS

The invention was made with Government support under Grant No. DK-44319 awarded by the National Institutes of Health. The Government has certain rights to the invention.

### SEQUENCE LISTING

The instant application contains a Sequence Listing which has been filed electronically in ASCII format and is hereby incorporated by reference in its entirety. Said ASCII copy, created on Aug. 29, 2018, is named 043214-086001USPX_SL.txt and is 8,653 bytes in size.

### FIELD OF INVENTION

Described herein are compositions, methods, and assays for the treatment and/or diagnosis of diseases and disorder involving oxazole-containing compounds.

### BACKGROUND OF THE INVENTION

Inflammatory bowel disease (IBD) is a complex disorder influenced by environmental and microbial factors in a genetically susceptible host, resulting in chronic relapsing and remitting inflammation of the gastrointestinal tract. Recent genome-wide association studies have revealed nearly 200 susceptibility loci associated with risk of IBD, including Crohn's Disease and ulcerative colitis, yet these studies further emphasize that genetic susceptibility is not sufficient for disease, and pathogenesis is likely triggered through host interactions with environmental stimuli.

### SUMMARY OF THE INVENTION

The identification and structural and molecular characterization of interactions between environmental factors with cellular systems of mucosal defense remains an underdeveloped source for therapies against IBD. As described herein, using in silico screens combined with experimental validation, we have identified a novel class of environmental ligands abundant in diet, microbes and industrial agriculture referred to herein as “oxazole-containing (OxC) compounds.” As shown herein, these OxC compounds have the capacity to induce symptoms in mammals consistent with the development of IBD, including massive bowel wall edema and dense infiltration of the superficial mucosal layers with polymorphonuclear granulocytes, together with the ulceration of the epithelial cell layer. As described herein, we have further defined structural features responsible for colitogenic activity and demonstrated that this class of compounds directs conserved cellular responses in epithelial cells resulting in altered inflammatory responses associated with IBD development. Thus, provided herein are novel compositions and targeted therapies for neutralizing the activity of OxC compounds to prevent or delay onset of epithelial barrier dysfunction and chronic inflammation associated with colitis.

In addition, based on our findings described herein, we determined that common structural elements of these compounds define a class of environmental stimuli that trigger defects in epithelial barrier protection, thereby promoting the development of colitis through a pathway that is derived from CD1d-restricted NKT cells. As described herein, our computational models indicate that the oxazole containing compounds (OxC compounds) are a novel class of molecules recognized by a ligand activated transcription factor, the aryl hydrocarbon receptor (AhR). AhR is an important sensor of polycyclic aromatic hydrocarbons, which mediates toxic effects of environmental xenobiotics, but can also direct development and function of specific immune subsets involved in mucosal immunity and autoimmune disease. The experimental evidence described herein also indicates that OxC compounds elicit cellular responses that modulate specific epithelial transcriptional targets and influence NKT cell immunomodulatory function, in an AhR dependent manner, providing a mechanistic link between exposure to “oxazolone-like” chemicals in the environment and epithelial barrier defects, mucosal dysbiosis and intestinal inflammation observed in IBD.

Accordingly, in some aspects, provided herein are pharmaceutical composition comprising an inhibitor of an Oxazole containing (OxC) compound and a pharmaceutically acceptable carrier, wherein the OxC compound is a compound of any of

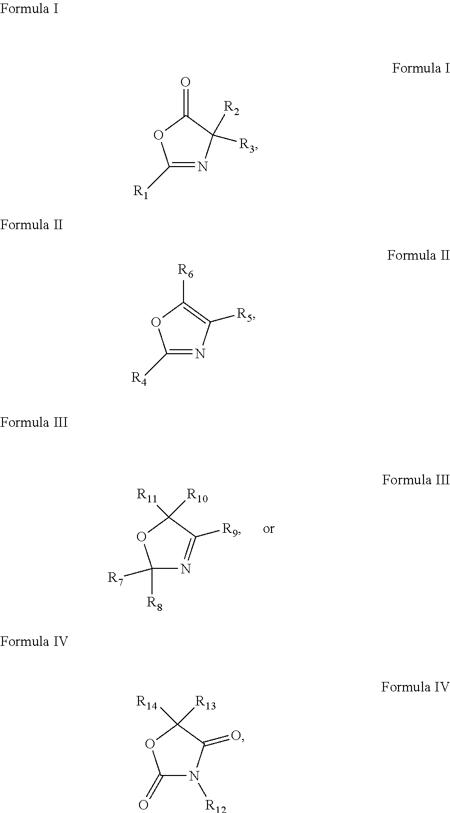

wherein R₁-R₁₄ are each independently selected from the group consisting of hydrogen, alkyl, alkenyl, alkynyl, aryl, heteroaryl, alkoxy, amino, and carbonyl, provided that each of Formulas I-IV has two or more R groups which are not hydrogen.

In some embodiments of these aspects and all such aspects described herein, the pharmaceutical composition of claim 1, wherein the compound of Formula I is Oxazolone

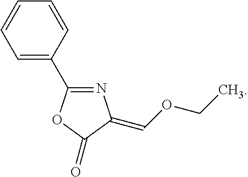 [SMILES: CCO/C=C1\N=C(c2ccccc2)OC1=O]

In some embodiments of these aspects and all such aspects described herein, the compound of Formula II is selected from

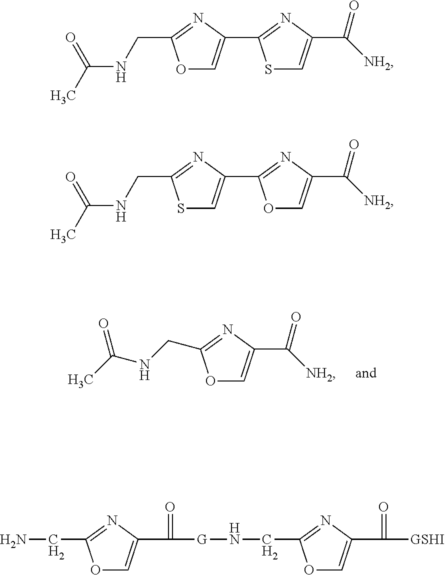

“GSHI” is disclosed as SEQ ID NO: 2.

In some embodiments of these aspects and all such aspects described herein, the compound of Formula III is 2,4,5-trimethyl-2,5-dihydro-1,3-oxazole (TMO):

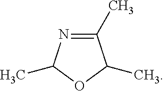

In some embodiments of these aspects and all such aspects described herein, the compound of Formula IV is vinclozolin:

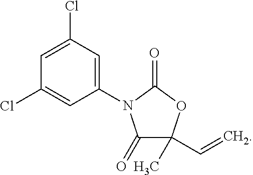 [SMILES: C=CC1(C)OC(=O)N(c2cc(Cl)cc(Cl)c2)C1=O]

In some embodiments of these aspects and all such aspects described herein, the OxC compound of Formula II is a thiazole/oxazole-modified microcin (TOMM).

In some embodiments of these aspects and all such aspects described herein, the TOMM is microcin B17 or mutants or fragments thereof.

In some embodiments of these aspects and all such aspects described herein, the inhibitor of an OxC compound specifically binds to the OxC compound, its metabolites, or a metabolic product induced by an OxC compound.

In some embodiments of these aspects and all such aspects described herein, the inhibitor of an OxC compound specifically binds to the OxC compound, its metabolites, or a metabolic product induced by an OxC compound and inhibits or prevents binding of the OxC compound to the Aryl Hydrocarbon Receptor (AhR) of SEQ ID NO: 1 and its activation.

In some embodiments of these aspects and all such aspects described herein, the inhibitor of an OxC compound inhibits or prevents binding of the OxC compound, its metabolites, or a metabolic product induced by an OxC compound, to one or more amino acids selected from H291, F295, S365, and Q383, thereby inhibiting AhR binding to an OxC compound and its activation.

In some embodiments of these aspects and all such aspects described herein, the inhibitor of an OxC compound is an Aryl Hydrocarbon Receptor (AhR) antagonist.

In some embodiments of these aspects and all such aspects described herein, the AhR antagonist binds to the Aryl Hydrocarbon Receptor (AhR) of SEQ ID NO: 1 at one or more amino acids selected from H291, F295, S365, and Q383 of SEQ ID NO: 1, and inhibits or prevents AhR binding to an OxC compound, its metabolites, or a metabolic product induced by an OxC compound.

In some embodiments of these aspects and all such aspects described herein, the inhibitor of an OxC compound is an antibody or antigen-binding fragment thereof.

In some embodiments of these aspects and all such aspects described herein, the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)2 fragments, a single chain antibody molecule, a diabody or a linear antibody.the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)2 fragments, a single chain antibody molecule, a diabody or a linear antibody.

In some embodiments of these aspects and all such aspects described herein, the inhibitor of an OxC compound is a small molecule.

In some aspects provided herein are methods of treatment of a disease or disorder associated with epithelial barrier integrity and/or iNKT cell-mediated inflammatory responses, comprising administering to a subject in need thereof a therapeutically effective amount of a pharmaceutical composition comprising an inhibitor of an Oxazole containing (OxC) compound and a pharmaceutically acceptable carrier, wherein the OxC compound is a compound of any of Formula I

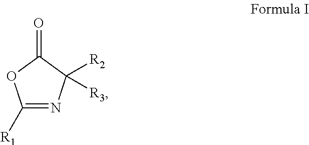

### Formula II

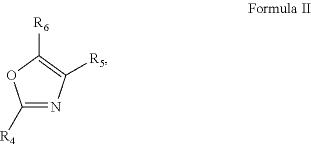

### Formula III

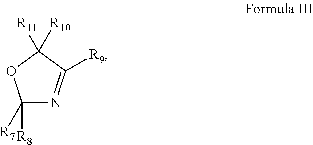

### Formula IV

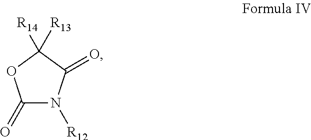

wherein R₁-R₁₄ are each independently selected from the group consisting of hydrogen, alkyl, alkenyl, alkynyl, aryl, heteroaryl, alkoxy, amino, and carbonyl, provided that each of Formulas I-IV has two or more R groups which are not hydrogen.

In some embodiments of these aspects and all such aspects described herein, the pharmaceutical composition of claim 1, wherein the compound of Formula I is Oxazolone

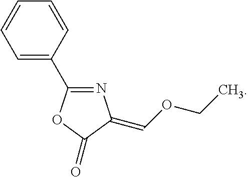 [SMILES: CCO/C=C1\N=C(c2ccccc2)OC1=O]

In some embodiments of these aspects and all such aspects described herein, the compound of Formula II is selected from

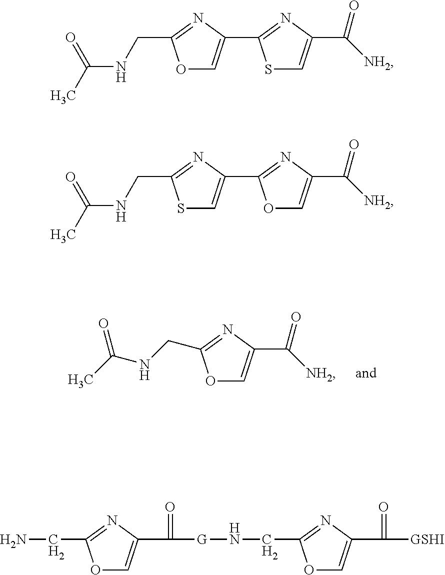

“GSHI” is disclosed as SEQ ID NO: 2.

In some embodiments of these aspects and all such aspects described herein, the compound of Formula III is 2,4,5-trimethyl-2,5-dihydro-1,3-oxazole (TMO):

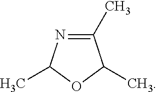

In some embodiments of these aspects and all such aspects described herein, the compound of Formula IV is vinclozolin:

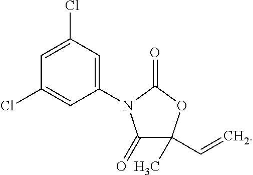 [SMILES: C=CC1(C)OC(=O)N(c2cc(Cl)cc(Cl)c2)C1=O]

In some embodiments of these aspects and all such aspects described herein, the OxC compound of Formula II is a thiazole/oxazole-modified microcin (TOMM).

In some embodiments of these aspects and all such aspects described herein, the TOMM is microcin B17 or mutants or fragments thereof.

In some embodiments of these aspects and all such aspects described herein, the inhibitor of an OxC compound specifically binds to the OxC compound, its metabolites, or a metabolic product induced by an OxC compound.

In some embodiments of these aspects and all such aspects described herein, the inhibitor of an OxC compound specifically binds to the OxC compound, its metabolites, or a metabolic product induced by an OxC compound and inhibits or prevents binding of the OxC compound to the Aryl Hydrocarbon Receptor (AhR) of SEQ ID NO: 1 and its activation.

In some embodiments of these aspects and all such aspects described herein, the inhibitor of an OxC compound inhibits or prevents binding of the OxC compound, its metabolites, or a metabolic product induced by an OxC compound to one or more amino acids selected from H291, F295, S365, and Q383, thereby inhibiting AhR binding to an OxC compound and its activation.

In some embodiments of these aspects and all such aspects described herein, the inhibitor of an OxC compound is an Aryl Hydrocarbon Receptor (AhR) antagonist.

In some embodiments of these aspects and all such aspects described herein, the AhR antagonist binds to the Aryl Hydrocarbon Receptor (AhR) of SEQ ID NO: 1 at one or more amino acids selected from H291, F295, S365, and Q383 of SEQ ID NO: 1, and inhibits or prevents AhR binding to an OxC compound, its metabolites, or a metabolic product induced by an OxC compound.

In some embodiments of these aspects and all such aspects described herein, the inhibitor of an OxC compound is an antibody or antigen-binding fragment thereof.

In some embodiments of these aspects and all such aspects described herein, the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)2 fragments, a single chain antibody molecule, a diabody or a linear antibody the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)2 fragments, a single chain antibody molecule, a diabody or a linear antibody.

In some embodiments of these aspects and all such aspects described herein, the inhibitor of an OxC compound is a small molecule.

In some embodiments of these aspects and all such aspects described herein, the disease or disorder associated with epithelial barrier integrity and/or iNKT cell-mediated inflammatory responses is an inflammatory bowel disease.

In some embodiments of these aspects and all such aspects described herein, the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

Provided herein in other aspects are assays for detecting the presence of an Oxazole containing (OxC) compound in a biological sample comprising measuring a level of a TOMM in a biological sample obtained from a subject, wherein if the level of an OxC compound is increased at least 1.5 fold relative to a control sample, the biological sample is identified as containing an OxC compound.

Provided herein in some aspects are assays for detecting the presence of an in a biological sample comprising measuring a level of a TOMM in a biological sample obtained from a subject, wherein if the level of a TOMM is increased at least 1.5 fold relative to a control sample, the biological sample is identified as containing a TOMM.

In some embodiments of these aspects and all such aspects described herein, the biological sample is a fecal, sputum, urine, or skin sample.

In some embodiments of these aspects and all such aspects described herein, the assays further comprise a step of obtaining the biological sample from the subject.

In some embodiments of these aspects and all such aspects described herein, the biological sample is obtained from a subject in need of treatment for IBD, or a subject at risk of or at increased risk of developing IBD.

In some embodiments of these aspects and all such aspects described herein, the measuring of the level of a TOMM in the biological sample comprises mass spectrometry, PCR, or an immunoassay.

In some embodiments of these aspects and all such aspects described herein, if the biological sample is identified as containing an OxC compound or a TOMM, the assay further comprises the step of administering any of the pharmaceutical compositions comprising an inhibitor of an Oxazole containing (OxC) compound and a pharmaceutically acceptable carrier described herein.

### Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein, an “Oxazole containing compound” or “OxC compound” refers to a compound of any of Formula I-IV having an oxazolone core (Formula I), an oxazole core (Formula II), an oxazoline core (Formula III), or a 2,4-oxazolidone core (Formula IV), and having the functional property of inducing one or more in vitro, ex vivo, or in vivo phenotypes associated with a colitis phenotype, as described herein. More specifically, OxC compounds have one or more of the functional properties of inducing or eliciting cell intrinsic responses and modulation of specific gene targets involved in iNKT cell inflammatory responses. Such responses induced or elicited by OxC compounds include, but are not limited to, the ability to: (i) elicit one or more changes (increase or decrease) in transcription and/or translation of gene targets in epithelial cells and/or iNKT cells, such as one or more of Mttp, Cxcl16, CD1d, and/or Hspl10; (ii) alter CD1d-restricted iNKT inflammatory responses , including iNKT cell immunomodulatory function; (iii) elicit one or more changes (increase or decrease) in transcription and/or translation of AhR gene targets, such as Cyp1a1 or IDO1; and/or (iv) induce colitis phenotype(s) in vivo, such as increased weight loss, colon shortening, histopathology (based on blind scoring by a pathologist), and/or expression of one or more cytokines characteristic of IBD.

As used herein, a “metabolite of an OXC compound” or a “metabolite induced by an OxC compound,” is a compound that is formed or induced when the OxC compound is metabolized, such as, for example, tryptophan. The term “active metabolite” refers to a biologically active compound that is formed when the OxC compound is metabolized. The term “metabolized,” as used herein, refers to the sum of the processes (including, but not limited to, hydrolysis reactions and reactions catalyzed by enzymes) by which a particular substance is changed by an organism. Thus, enzymes can produce specific structural alterations to a compound. Further information on metabolism can be obtained from Goodman and Gilman's The Pharmacological Basis of Therapeutics, Twelfth Edition (2011). Metabolites of and metabolites induced by the compounds disclosed herein can be identified either by administration of OxC compounds to a subject and analysis of tissue and/or fluid samples from the subject, or by incubation of compounds with cells in vitro and analysis of the resulting compounds.

As used herein, an “inhibitor of an OxC compound” or “OxC compound inhibitor” refers to an agent or compound that inhibits one or more processes, mechanisms, effects, responses, functions, activities or pathways mediated by an OxC compound, its metabolites or a metabolic product induced by the OxC compound binding to an endogenous receptor, such as the AhR receptor. Such processes, mechanisms, effects, responses, functions, activities or pathways include, for example, functional properties of inducing or eliciting cell intrinsic responses and modulation of specific gene targets involved in iNKT cell inflammatory responses.

The term “agent” as used herein in reference to an inhibitor of an OxC compound means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities.

As used herein, “selectively binds” or “specifically binds” refers to the ability of an inhibitor of an OxC compound described herein to bind to a target, such as an OxC compound of any of Formulas (I)-(IV), is metabolites or a metabolic product induced by the OxC compound. with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less.

As used herein, an “AhR antagonist” refers to an inhibitor of an OxC compound, its metabolites, or a metabolic product induced by the OxC compound that does not provoke a biological response itself upon specifically binding to the AhR polypeptide or polynucleotide encoding the AhR, but blocks or dampens OxC compound-mediated responses, i.e., an AhR antagonist can bind but does not activate the AhR polypeptide or polynucleotide encoding the AhR, and the binding disrupts the interaction, or displaces an OxC compound, its metabolites, or a metabolic product induced by the OxC compound and/or inhibits the function of an OxC compound, its metabolites, or a metabolic product induced by the OxC compound binding to the AhR.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the phrase “a disease or disorder associated with epithelial cell barrier integrity” refers to any disease or disorder associated with or known to be caused, at least in part, by defects in epithelial cell barrier integrity or function. As particular nonlimiting examples, the conditions with which the epithelial cell barrier dysfunction occurs include inflammatory bowel disease, Crohn's disease, dermatitis, including allergic (contact dermatitis, such as irritant dermatitis, phototoxic dermatitis, allergic dermatitis, photoallergic dermatitis, contact urticaria, systemic contact-type dermatitis and the like and atopic dermatitis), as well as irritant dermatitis; gut-derived sepsis, a burn injury, a chemical contact injury, acute lung injury, asthma, COPD, neonatal necrotizing enterocolitis, severe neutropenia, toxic colitis, enteropathy, transplant rejection, pouchitis, pig-bel, uremic pericardial effusion, leakage in the vitreous of the eye, macular degeneration, retinal dysfunction, and infection (e.g., viral infection, bacterial infection, opportunistic bacterial infection, Clostridium dificile infection, Pseudomonas aeruginosa infection, Pseudomnonas-mediated ophthalmologic infection, Pseudomonas-mediated otologic infection and Pseudomonas-mediated cutaneous infection).

As used herein, the phrase “a disease or disorder associated with iNKT cells inflammatory responses” refers to any disease or disorder associated with or known to be caused, at least in part, by unwanted or excessive or increased iNKT cell immune responses, as the term is defined herein. Examples of diseases or disorders mediated by such iNKT cells include, but are not limited to, rheumatoid arthritis, systemic lupus erythematosus, type 1 diabetes, psoriasis, atherosclerosis, allergic asthma, graft versus host disease, haematological cancers.

As used herein, an “iNKT cell immune response” is a response by an iNKT cell to a stimulus, such as exposure to an OxC compound. Such responses by these cells can include, for example, cytotoxicity, proliferation, cytokine or chemokine production, and/or trafficking to a tissue site.

As used herein, the terms “subject” or “individual” or “animal” or “patient” or “mammal,” refer to any subject, particularly a mammalian subject, preferably a human subject, for whom diagnosis, prognosis, or therapy is desired.

As used herein, the terms “colitides,” “colitis,” “inflammatory bowel disease,” and “IBD” refer to inflammatory conditions of the colon and/or small intestine, often characterized by abdominal pain, vomiting, diarrhea, rectal bleeding, cramps, and/or anemia. Examples of IBD include Crohn's disease, ulcerative colitis, and various classifications of colitides, e.g., idiopathic colitides (e.g., microscopic colitis, lymphocytic colitis, and collagenous colitis), iatrogenic colitides (e.g., including that associated with antibiotic administration, diversion colitis, neutropenic enterocolitis, disinfectant colitis, corrosive colitis, nonsteroidal anti-inflammatory drug and salicylate-induced colitis, toxic epidermal necrolysis, and other chemical-induced colitides), ischemic colitis, infectious colitides (e.g., Clostridium difficile colitis), eosinophilic colitis. In some embodiments, the condition (e.g., the IBD) is chronic, acute, and/or recurring. In some embodiments, the condition is associated with antibiotic administration.

By “reduce” or “inhibit” is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. “Complete inhibition” is a 100% inhibition as compared to a reference level.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder.

The term “effective amount” as used herein refers to the amount of an inhibitor of an OxC compound needed to alleviate at least one or more symptom of the disease or disorder, and relates to a sufficient amount of pharmacological composition to provide the desired effect, for example, treating IBD. The term “therapeutically effective amount” therefore refers to an amount of an inhibitor of an OxC compound using the methods as disclosed herein, that is sufficient to effect a particular effect when administered to a typical subject.

As used herein, a “sample” or “biological sample” can refer to a solid, semi-solid or liquid sample, including, but not limited to, feces, sputum, urine, a tissue sample, a cellular sample, a cellular extract, plasma, serum, blood, cord blood, body secretions from the nose, oropharynx, gastrointestinal tract, bile or genitourinary tract, tissue biopsies of any organ, a tissue fluid such as cerebrospinal, occular or joint fluids, or any combination thereof.

As used herein, “determining the amount of an OxC compound,” or “measuring or quantifying the amount of of an OxC compound,” refers to any investigative or analytic method, procedure, or assay that can be used to for qualitatively assessing or quantitatively measuring the presence or amount of one or more OxC compounds, such as TOMMs, in a biological sample.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1F depicts in accordance with an embodiment of the invention, structures of oxazolone (1A), oxazolone-containing compound vinclozolin (1B), 2,4,5-trimethyl-2,5-dihydro-1,3-oxazole (TMO) (1C), AhR agonist ITE (1D), and control compounds 2-methyl-1-pyrroline (2-MP) (1E) and 1,2,4-trimethylcyclopentane (TMC) (1F).

FIGS. 2A-2B depict in accordance with an embodiment of the invention, oxazolone and oxazole-containing compounds which differentially regulate Natural Killer T cell signature genes. Mode-K cells were stimulated for 48 hours with the listed compounds and MU_{T}) (2A) and Cxcl16 (2B) transcript express was quantified by qPCR normalized to β-actin, a housekeeping gene. Graph color coded to indicate control, OxC, and ITE compounds.

FIGS. 3A-3B depict in accordance with an embodiment of the invention, (3A) MODE-K cells transfected with control- or AhR-targeted siRNA. After 48 hours, these cells were stimulated with the compounds indicated for 48 hours. Cells were washed and incubated with alpha-galactosylceramide and subsequently co-cultured with the 24.7 NKT hybridoma. IL-10 production was measured by ELISA. (3B) Hepatocytes from WT or AhR-deficient mice were stimulated as in (3A).

FIGS. 4A-4C depict in accordance with an embodiment of the invention, (4A) MODE-K cells stimulated with the indicated control or OxC compounds and CD1d transcript production was measured by quantitative real-time PCR, normalized to (3-actin. (4B) CD1d intracellular and surface protein expression was measured using a specific CD1d antibody by flow cytometry. (4C) MODE-K cells conditioned with the indicated vehicle, control or OxC compounds for 48 hours and cultured with α-galactosylceramide and surface CD1d:lipid antigen presentation was measured using specific antibody by flow cytometry.

FIG. 5 depicts in accordance with an embodiment of the invention, animals (n=5) pre-sensitized with 3% oxazolone (Ox) or TMO and 5 days later administered oxazolone, TMO, or vehicle. Colitis scores were blindly performed by a pathologist.

FIGS. 6A-6B depict in accordance with an embodiment of the invention, animals (n=5) pre-sensitized with 3% oxazolone or TMO and 5 days later administered oxazolone, TMO. Distal colon explants were cultured overnight and IL-10 (6A) and IL-13 (6B) production was measured by ELISA.

FIGS. 7A-7B depict in accordance with an embodiment of the invention, (7A) structure of the OxC compound, B17 microcin. (7B) MODE-K stimulated with DMSO, oxazolone or lysates from an E. coli strain (recA56) expressing or not expressing B17. Cells were incubated with aGC and co-cultured with24.7 iNKT hybridoma and IL-10 production was measured. “VGIGGGGGGGGG”, “G2QG2”, “GGNG”, and “GSHI” are disclosed as SEQ ID NO: 3, SEQ ID NO: 6, SEQ ID NO: 5, and SEQ ID NO: 2, respectively.

FIGS. 8A-8C depict in accordance with an embodiment of the invention, MODE-K transfected with control or AhR-targeted siRNA. After 48 hours, MODE-K cells were stimulated with the indicated compounds. Cyp1a1 (8A) or IDO1 (8B, 8C) were quantified by qPCR.

FIGS. 9A-9B depict in accordance with an embodiment of the invention, predicted ligand binding orientation of ITE (9A), oxazolone and TMO (9B) within the human aryl hydrocarbon receptor PAS B domain. Homology structure was built using HIF-2a PAS B domain template.

FIG. 10 depicts in accordance with an embodiment of the invention, synthetic oxazole analogs and fragments of Microcin B17.“GSHI” is disclosed as SEQ ID NO: 2.

FIG. 11 depicts in accordance with an embodiment of the invention, Microcin B17 produced by wild-type and mutant strains of E. coli. “VGI[G]₉”, “GGNGG”, “GGNG”, “GSHI”, and “GGQGG” are disclosed as SEQ ID NO: 3, SEQ ID NO: 4, SEQ ID NO: 5, SEQ ID NO: 2, and SEQ ID NO: 6, respectively.

FIG. 12 depicts in accordance with an embodiment of the invention, that responses to OxC compounds elicit Th2 responses and are CD1d dependent.

FIG. 13 depicts in accordance with an embodiment of the invention, that IL-10 production by NKT cells is diminished after culture with oxazolone, TMO or subset of synthetic microcin compounds.

FIG. 14 depicts in accordance with an embodiment of the invention, that Oxazolone, TMO and a subset of synthetic microcin peptides activate an AhR reporter, but not an NFkB reporter.

FIG. 15 depicts in accordance with an embodiment of the invention, that epithelial specific deficiency provides protection against oxazolone colitis.

FIG. 16 depicts in accordance with an embodiment of the invention, that tryptophan depletion alters AhR activation and CD1d mediated antigen presentation in MODE-K cells.

FIG. 17 depicts in accordance with an embodiment of the invention, microcin and microcin synthetic analogs that target bacterial gyrases. “VGI[G]₉”, “GGNGG”, “GGNG”, and “GSHI” are disclosed as SEQ ID NO: 3, SEQ ID NO: 4, SEQ ID NO: 5, and SEQ ID NO:2, respectively.

FIG. 18 depicts lessons from the oxazolone model of inflammation. As described herein, the oxazolone model of inflammation manifests histopathological features of Ulcerative Colitis, based on a proposed mechanism of acting as a “haptenating agent.” The oxazolone model of inflammation Induces Th2 and/or Th1 responses in genetically susceptible hosts (Boirevant et al. 1998; Heller et al. 2002, Iijima et al. 2004). Inflammation primarily mediated through CD1d-restricted invariant NKT cell activity (Heller et al. 2002), although iNKT frequency does not change during course of inflammation/recovery. Microsomal Triglyceride Transfer Protein (MTP) regulated CD1d responses on epithelial cells alleviate inflammation through induction of IL-10 (Brozovic et al. 2004; Dougan et al. 2005, 2007; Zeissig et al. 2010; Olszak et al. 2014).

FIG. 19 depicts estimated exposure to oxazole-containing compounds as derived from ToxCast and Tox21 chemical databases.

FIG. 20 depicts a larger class of compounds that promote colitis in genetically susceptible hosts, which includes oxazolone as a representative.“VGIGGGGGGGGG”, “G2QG2”, “GGNG”, and “GSHI” are disclosed as SEQ ID NO: 3, SEQ ID NO: 6, SEQ ID NO: 5, and SEQ ID NO: 2, respectively.

FIG. 21 demonstrates oxazole compounds regulate expression of iNKT signature genes in epithelial cells.

FIG. 22 demonstrates OxC compounds do not modulate CD1d expression or trafficking.

FIG. 23 demonstrates OxC compounds alter CD1d lipid antigen presentation.

FIG. 24 demonstrates conditioning of MODE-K cells with OxC compounds attenuates CD1d-dependent invariant/noninvariant murine NKT responses.

FIG. 25 demonstrates oxazole compounds manifest histological features of oxazolone colitis.

FIG. 26 demonstrates host-mediated inflammatory response to oxazole compounds is CD1d-dependent and does not require a priori antigen stimulation.

FIG. 27 demonstrates OxC compounds elicit Th2 response in vivo that is dependent on CD1d and the presence of iNKT cells.

FIG. 28 shows Microcin B17 is encoded by the TOMM (thiazole/oxazol-modified microcins) class of operon: a DNA gyrase inhibitor.

FIG. 29 shows oxazole rings are prevalent in the microbial world. Kelly, Nat Chem Biol Rev 2012. “VGIGGGGGGGGG”, “G2QG2”, “GGNG”, and “GSHI” are disclosed as SEQ ID NO: 3, SEQ ID NO: 6, SEQ ID NO: 5, and SEQ ID NO: 2, respectively.

FIG. 30 demonstrates host response to Frag-oz synthetic microbial analog is exacerbated in the absence of microbes.

FIG. 31 demonstrates CD1d regulated Host IL-10 response to Frag-oz synthetic microbial analog. 19G11/IgG2b (50 ug/g) injected I.P. (−2, −1) followed by injection of 1% SIM 195 (50%EtOH) followed 12 hours later by final 19G11/IgG2b injection. Colon harvested day 2 after SIM195 treatment and epithelial compartment collected by mucosal scrapings. Lamina propria compartment isolated after HBSS/1 mMEDTA wash. Fractions homogenized in PBS supplmented with protease inhibitor cocktail.

FIG. 32 demonstrates oxazole compounds activate the AhR pathway in intestinal epithelial cells and that the AhR is a putative target of oxazole compounds.

FIG. 33 demonstrates Vil-Cre+X AhR fl/fl mice are protected against oxazolone colitis. Mice presensitized with 3% oxazolone (100%ETOH) followed by intra-rectal adminstration of 1% oxazolone (50%EtOH). N=9-11

FIG. 34 demonstrates synthetic microcin analog Frag-oz induces colitis phenotype associated with CD1d-dependent Th1/Th2 response.

### DETAILED DESCRIPTION OF THE INVENTION

Provided herein are novel compositions, targeted therapeutic methods, and assays for neutralizing and/or inhibiting the activity of “oxazole-containing (OxC) compounds,” to prevent or delay the onset of epithelial barrier dysfunction and chronic inflammation associated with various disorders, such as colitis.

### Oxazole Containing (OxC) Compounds

Oxazoles represent a class of highly reactive heterocyclic aromatic organic compounds highly abundant in natural and synthetic compounds present in diet and agriculture, and can be produced by a diverse group of microbial species, including those in human mucosal systems. Oxazoles adopt a 5-membered ring structure featuring oxygen and nitrogen at the 1- and 3-positions following Hantzsch-Wedman nomenclature and are closely related to the β-lactam ring systems found in penicillin. The oxazole ring contains numerous reactive sites allowing for a wide variety of transformations, making it a popular industrial substrate for synthetic production of heterocyclic compounds, including amino acid synthesis. Oxazole-containing (OxC) compounds are also a component of many food systems including coffee, cocoa, barley, potato and meat products, and have been appropriated as an additive to fruits and vegetables as a pesticide. For instance, 55,000 pounds of vinclozolin, an OxC compound with anti-fungal properties, was used to treat crops annually in the U.S. according to recent estimates. The microbial world is also an abundant source of oxazole as part of a structurally diverse class of ribosomally derived peptides dubbed “thiazole/oxazole-modified microcins” (TOMMs), generated through post-translation installation of heterocylcles derived from cysteine, serine, and threonine residues and widely disseminated across the phylogenetic spectra of bacterial secretion systems, including commensals such as Proteobacteria, associated with IBD pathogenesis. TOMM products are functionally diverse with anti-microbial properties and thus selectively endow ecological advantages. In addition, similar products are generated by other genera of microbes that are associated with other inflammatory diseases including those of the skin (e.g., Corynebacteria). For instance, many Escherichia coli (E. coli) strains encode a 7 gene operon that directs production of an oxazole-ring containing heterocyclic peptide, termed B17, which inactivates bacterial DNA gyrases. The antimicrobial capacity of oxazole rings has been harnessed through isolation and synthetic production of antibiotics such as sulfamethyloxazole. The studies described herein reveal that TOMM products are not only important in antimicrobial defense among competing bacterial strains, but also have a previously unappreciated inflammatory activity on the host.

Though precise measurements of the human rate of exposure to OxC compounds are sorely lacking, heuristic methods to predict potential human exposure to a select group of OxC compounds found in urine samples estimate exposures in the range of 10⁻⁷g/kg of body weight per day. These estimates do not include the content derived from pathogenic (or pathobiotic) microbes, which our studies indicate can be a major source. Detecting them and preventing their interactions with the host is therefore extremely important, as shown herein.

A prototype compound 4-ethoxymethylene-2-phenyl-oxazol-5-one (oxazolone) is known to possess colitogenic potential through a mechanism dependent on CD1d, a non-classical major histocompatibility complex-like molecule that presents lipid antigens to invariant natural killer T (iNKT) cells, proposed to be a major source of inflammatory cytokines in IBD. Classically, oxazolone has been proposed to act as a haptenating agent, suggesting that re-exposure to this “antigen” might trigger inflammatory responses. However, no direct evidence of oxazolone-peptide or oxazolone-lipid complexes in colonic mucosa has been detected.

Instead, as demonstrated herein, we have surprisingly discovered that oxazolone elicits cell intrinsic responses and modulates specific gene targets involved in NKT cell biology, independent of any direct antigenic properties. Oxazolone contains 4 major functional groups: a 5 membered oxazole ring, 2′ phenyl conjugate, 4′ethoxymethylene, and 5′-ketone. In order to isolate the cellular activity of oxazolone, we interrogated epithelial cells with compounds containing specific deletions/substitutions of key components within the oxazolone structure. Accordingly, as shown herein, a natural dietary component, 2,4,5-trimethyl-2,5,-dihydro-1,3-oxazole (TMO), found in coffee and other food sources which lacks the 2-phenyl ring, 5-ketone, and 4-ethyoxymethylene, was identified as the minimal structural-unit sufficient to confer cellular activity in epithelial cells based on its ability to elicit changes in transcription of specific gene targets in epithelial cells and alter CD1d-restricted iNKT inflammatory responses in a similar manner to oxazolone. As demonstrated herein, these cellular responses were dependent on the presence of both the oxygen and nitrogen occupying the 1′ and 3′ positions, respectively as neither 1-methyl-pyrroline (MP) nor 1,2,4-trimelitylcyclopentane (TMC) induced transcriptional effects like oxazolone or TMO. Furthermore, as shown herein, the isolated oxazole structure is sufficient to induce a colitis phenotype in vivo, as administration of TMO phenocopies the inflammation associated with oxazolone and leads to increased weight loss, colon shortening, histopathology (based on blind scoring by a pathologist) and expression of cytokines characteristic of IBD compared to vehicle control animals. We then investigated the broader capacity of OxC compounds to stimulate epithelial transcription changes and influence CD1d-restricted iNKT cytokine production. Vinclozolin, a ubiquitous fungicide detectable in human urine, demonstrates a similar transcriptional pattern and exerts modulatory effects on iNKT cell responses similar to oxazolone. Likewise stimulation of lysates from an E. coli strain that produces the OxC B17 peptide also altered CD1d restricted iNKT cytokine production compared to the same strain lacking the B17 operon.

Accordingly, the data presented herein support a model in which exposure to a broad class of oxazole-ring-containing compounds can direct profound transcriptional changes in responsive tissues, such as the epithelium, that concomitantly influence barrier integrity at mucosal sites and modulate iNKT cell inflammatory responses associated with colitis. Based on these results, a major source of OxC compounds is a class of microbial-derived TOMMs, such as the B17 microcin, secreted by pathobiontic commensal bacteria to establish their ecological niche making the microbial source of molecules either in the gut, skin, or other body surfaces the greatest source of these potentially inflammatory compounds, Interestingly, though there is great diversity in sequence identity and microbial function of individual TOMMs, the biosynthetic pathway contains at least 3 genes, encoding a cyclodehydratase, a dehydrogenase and docking protein, respectively, that possess 10-22% amino acid sequence conservation across species.

### Inhibitors of OxC Compounds

Accordingly, provided herein, in some aspects, are pharmaceutical compositions comprising an inhibitor of an OxC compound and a pharmaceutically acceptable carrier.

As used herein, an “Oxazole containing compound” or “OxC compound” refers to a compound of any of Formula I-IV having an oxazolone core (Formula I), an oxazole core (Formula II), an oxazoline core (Formula III), or a 2,4-oxazolidone core (Formula IV), and having the functional property of inducing one or more in vitro, ex vivo, or in vivo phenotypes associated with a colitis phenotype, as described herein. More specifically, OxC compounds have one or more of the functional properties of inducing or eliciting cell intrinsic responses and modulation of specific gene targets involved in iNKT cell inflammatory responses. Such responses induced or elicited by OxC compounds include, but are not limited to, the ability to: (i) elicit one or more changes (increase or decrease) in transcription and/or translation of gene targets in epithelial cells and/or iNKT cells, such as one or more of Mttp, Cxcl16, CD1d, and/or Hsp110; (ii) alter CD1d-restricted iNKT inflammatory responses , including iNKT cell immunomodulatory function; (iii) elicit one or more changes (increase or decrease) in transcription and/or translation of AhR gene targets, such as Cyp1a1 or IDO1; and/or (iv) induce colitis phenotype(s) in vivo, such as increased weight loss, colon shortening, histopathology (based on blind scoring by a pathologist), and/or expression of one or more cytokines characteristic of IBD.

Assays and methods to identify in vitro, ex vivo, or in vivo functional properties of an OxC compound are provided herein, e.g., in the Examples. For example, epithelial (e.g., MODE-K) and myeloid cell lines (e.g., JAWSII, RAW264.7) are stimulated with a putative OxC compound and production of NKT signatures and CD1d-regulated epithelial barrier associated gene targets (Mttp, Cxcl16, CD1d, Hsp110) are assessed and oxazolone conditioning of CD1d-bearing epithelial or myeloid APCs on NKT cell responses determined, using both invariant, auto-reactive, or non-variant NKT hybridomas in the presence of absence of a CD1d lipid antigen, by measuring proinflammatory (IL-4, IL-13, IFN γ) and anti-inflammatory (IL-10) cytokine production. Such responses can be compared to those elicited by known OxC control compounds, such as oxazolone and/or TMO. To assess cellular effects of OxC on NKT activity, primary iNKT cells isolated from colonic LP or peripheral organs are conditioned with OxC or control compounds and co-cultured with MODE-K, JAWSII, or RAW264.7 cells +/−α-GC and cytokine production is measured. To assess, for example, in vivo tissue-specific cellular pathways leading to disease pathogenesis, animals can be subjected to both acute and chronic regimens of a putative OxC compound and aspects of colitis development monitored: weight loss, histopathology, immunological response, and cellular composition of the colonic lamina propria. Colons can be subjected to blind quantitative histopathology scoring using the following criteria: loss of goblet cells, presence of crypt abscesses, hyperemia in the mucosa, cellular infiltration in the lamina propria elongation of colonic mucosa, and epithelial erosion. The number of conventional T cell, NKT cell and mucosal APC subsets can be enumerated by flow cytometry. At empirically determined time points, colonic epithelial and lamina propria mononuclear cells can be sorted by FACS and subjected to quantitative real-time PCR analysis for tissue specific OxC targets, including, for example, Mttp and Cxcl16.

As used herein, a “metabolite of an OXC compound” or a “metabolite induced by an OxC compound,” is a compound that is formed or induced when the OxC compound is metabolized, such as, for example, tryptophan. The term “active metabolite” refers to a biologically active compound that is formed when the OxC compound is metabolized. The term “metabolized,” as used herein, refers to the sum of the processes (including, but not limited to, hydrolysis reactions and reactions catalyzed by enzymes) by which a particular substance is changed by an organism. Thus, enzymes can produce specific structural alterations to a compound. Further information on metabolism can be obtained from Goodman and Gilman's The Pharmacological Basis of Therapeutics, Twelfth Edition (2011). Metabolites of and metabolites induced by the compounds disclosed herein can be identified either by administration of OxC compounds to a subject and analysis of tissue and/or fluid samples from the subject, or by incubation of compounds with cells in vitro and analysis of the resulting compounds.

As used herein, an “inhibitor of an OxC compound” or “OxC compound inhibitor” refers to an agent or compound that inhibits one or more processes, mechanisms, effects, responses, functions, activities or pathways mediated by an OxC compound binding to an endogenous receptor, such as the AhR receptor. Such processes, mechanisms, effects, responses, functions, activities or pathways include, for example, functional properties of inducing or eliciting cell intrinsic responses and modulation of specific gene targets involved in iNKT cell inflammatory responses. Such responses inhibited by an inhibitor of an OxC compound include, but are not limited to, the ability to: (i) elicit one or more changes (increase or decrease) in transcription and/or translation of gene targets in epithelial cells and/or iNKT cells, such as one or more of Mttp, Cxcl16, CD1d, and/or Hsp110; (ii) alter CD1d-restricted iNKT inflammatory responses , including iNKT cell immunomodulatory function; (iii) elicit one or more changes (increase or decrease) in transcription and/or translation of AhR gene targets, such as Cyp1a1 or IDO1; and/or (iv) induce colitis phenotype(s) in vivo, such as increased weight loss, colon shortening, histopathology (based on blind scoring by a pathologist), and/or expression of one or more cytokines characteristic of IBD.

The term “agent” as used herein in reference to an inhibitor of an OxC compound means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody or antigen-binding fragment thereof, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. Agents can be known to have a desired activity and/or property, e.g., inhibit an OxC compound, or can be selected from a library of diverse compounds, using, for example, the screening methods described herein.

In some embodiments of the aspects described herein, an OxC compound is a compound of any of Formulas I-IV.

Structures of OxC compounds of Formula I, Formula II, Formula III, or Formula IV comprise:

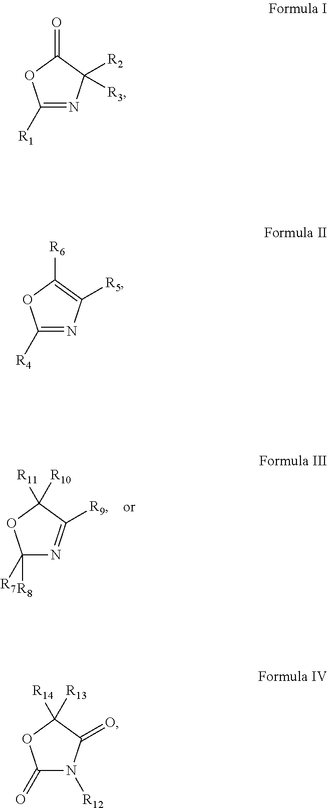

wherein R₁-R₁₄ are each independently selected from the group consisting of hydrogen, alkyl, alkenyl, alkynyl, aryl, heteroaryl, alkoxy, amino, and carbonyl, provided that each of Formulas I-IV has two or more R groups which are not hydrogen.

In some embodiments of the OxC compounds described herein, the alkyl, alkenyl, alkynyl, aryl, or heteroaryl may each be independently substituted with halogen, alkoxy, amino, or carbonyl.

In some embodiments of the OxC compounds described herein, R₁, R₄, R₅, R₇, R₈, or R₁₂ are each independently hydrogen, carbonyl, heteroaryl, unsubstituted aryl, or aryl substituted with halogen. In some embodiments of the OxC compounds described herein, R₁, R₄, R₅, R₇, R₈, or R₁₂ are each independently hydrogen, amide, thiazole, unsubstituted phenyl, or phenyl substituted with fluorine, chlorine, bromine, or iodine in one or more of the ortho, meta, or para positions.

In some embodiments of the OxC compounds described herein, R₂ and R₃ together form an alkenyl group substituted with alkoxy. In some embodiments of the OxC compounds described herein, R₁ is aryl, and R₂ and R₃ are alkyl or alkenyl. In In some embodiments of the OxC compounds described herein, R₄ is alkylamino, alkylamido, or substituted thiazole, R₅ is carbonyl or substituted thiazole, and R₆ is hydrogen. In some embodiments of the OxC compounds described herein, R₇, R₈, R₉, R₁₀, or R₁₁ are each independently hydrogen or alkyl. In some embodiments of the OxC compounds described herein, R₇ and R₁₀ are hydrogen and R₈, R₉, and R₁₁ are each alkyl selected from methyl, ethyl, propyl, and butyl. In some embodiments of the OxC compounds described herein, R₁₂ is halogen-substituted aryl, and R₁₃ and R₁₄ are each alkenyl or alkyl selected from methyl, ethyl, propyl, and butyl.

In some embodiments, the OxC compound is a compound of Formula I, for example Oxazolone:

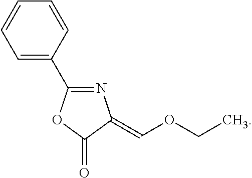 [SMILES: CCO/C=C1\N=C(c2ccccc2)OC1=O]

In some embodiments, the OxC compound is a compound of Formula II, for example

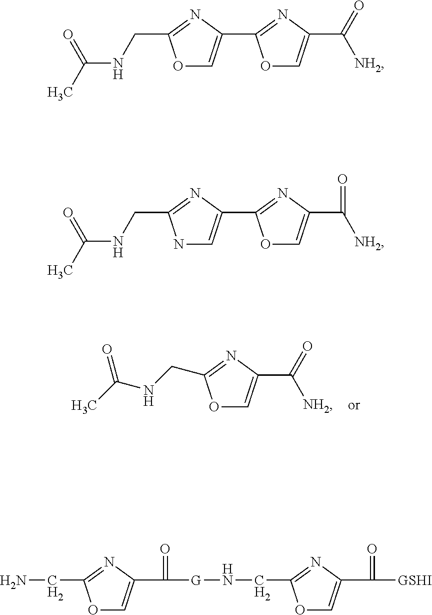

“GSHI” is disclosed as SEQ ID NO: 2.

In some embodiments, the OxC compound is a compound of Formula III, for example 2,4,5-trimethyl-2,5-dihydro-1,3-oxazole (TMO):

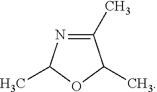

In some embodiments, the OxC compound is a compound of Formula IV, for example vinclozolin:

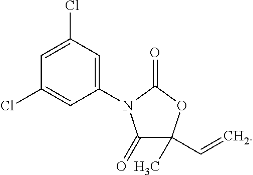 [SMILES: C=CC1(C)OC(=O)N(c2cc(Cl)cc(Cl)c2)C1=O]

In some embodiments, the OxC compound is a thiazole/oxazole-modified microcin (TOMM). In some such embodiments, the TOMM is microcin B17 or mutants or fragments thereof. Microcin B17 has the structure:

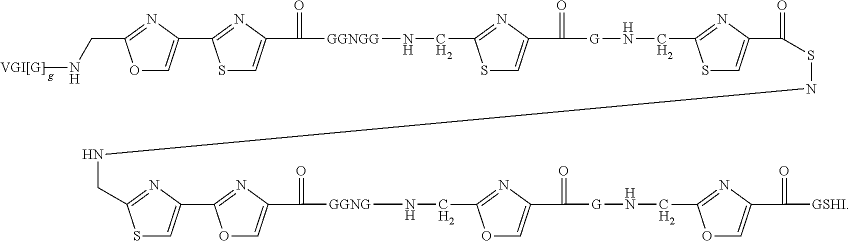

Mutants of Microcin B17 include Mcb/SHI:

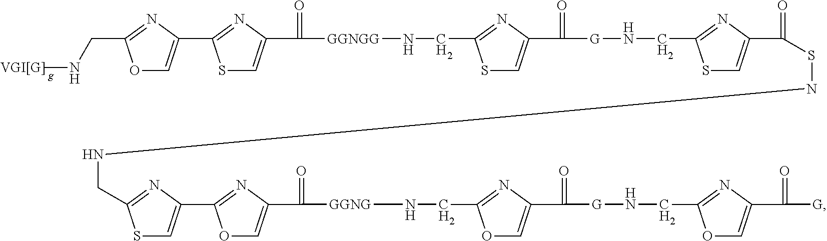

### Mcb Δ+1:

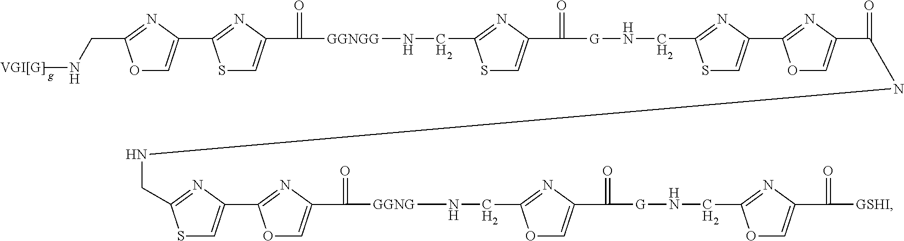

### and Mcbl:

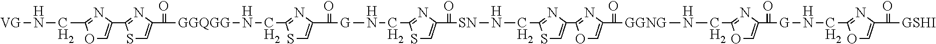

“VGI[G]9”, “GGNGG”, “GGNG”, “GSHI”, and “GGQGG” are disclosed as SEQ ID NO:3, SEQ ID NO: 4, SEQ ID NO: 5, SEQ ID NO: 2, and SEQ ID NO: 6, respectively.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C6alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula =CRₐR_{b}. CX alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH=CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y} aryl are typically used where X and Y indicate the number of carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thienop, 3-blpyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo [1,5-a]pyridine, pyrazolo[1,5]-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cxcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cxheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term “carbonyl” means the radical —C(O)—. It is noted that the carbonyl radical can be further substituted with a variety of substituents to form different carbonyl groups including aldehyde (e.g., formyl), acids, acid halides, amides, esters, ketones, and the like. In some embodiments, the carbonyl group is substituted with a heterocyclyl. For example, the carbonyl group can be in the form of an ester or amide when connected to an oxygen or nitrogen atom of heterocyclyl.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N^{-P}(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (-0—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring canbe such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)—O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino” —NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a CI alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} caneach independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In some embodiments, an inhibitor of an OxC compound specifically binds to the OxC compound. As used herein, “selectively binds” or “specifically binds” refers to the ability of an inhibitor of an OxC compound, described herein to bind to a target, such as an OxC compound of any of Formulas (I)-(IV), with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. Specific binding can be influenced by, for example, the affinity and avidity of the inhibitor and the concentration of the inhibitor used. The person of ordinary skill in the art can determine appropriate conditions under which the inhibitors described herein selectively bind using any suitable methods, such as titration of an inhibitor of an OxC compound in a suitable cell binding assay, such as those described herein.

With respect to the OxC compound, the term “interaction site” means a site, epitope, antigenic determinant, or part, that is a site for binding to a receptor, such as the AhR, or other binding partner, a site for allosteric interaction, which is involved in a biological action or mechanism of the OxC compound. More generally, an “interaction site” can be any site, epitope, antigenic determinant, or part, on the OxC compound to which an inhibitor described herein can bind, such that one or more processes, mechanisms, effects, responses, functions, activities or pathways mediated by an OxC compound binding to an endogenous receptor, such as the AhR receptor, is inhibited.

The terms “inhibit,” “decrease,” and “reduce”, are all used herein generally to mean a decrease by a statistically significant amount. Accordingly, inhibition is achieved when the activity value of the responses normally mediated by an OxC compound is about at least 10% less, at least 20% less, at least 30% less, at least 40% less, at least 50% less, at least 60% less, at least 70% less, at least 80% less, at least 90% less, at least 95% less, at least 98% less, at least 99% less, up to including 100% or less, i.e., absent, or undetectable, in comparison to a reference or control level in the absence of the OxC compound inhibitor.

As demonstrated herein, OxC compounds are a class of molecules recognized by the aryl hydrocarbon receptor (AhR). The experimental evidence described herein indicates that OxC compounds themselves and/or metabolic effects that induce or regulate Ahr ligands function in an AhR-dependent manner to elicit cellular responses that modulate specific epithelial transcriptional targets and influence NKT cell immunomodulatory function.

The Aryl Hydrocarbon Receptor (“AhR”) is a ligand-dependent member of the family of basic-helix-loop-helix transcription factors that has been found to be activated by numerous structurally diverse synthetic and naturally occurring compounds, such as polycyclic aromatic hydrocarbons, indoles, and flavonoids. In the absence of bound ligand, the AhR is present in a latent conformation in the cytoplasmic compartment of the cell associated with two molecules of the molecular chaperone heat shock protein 90 (“hsp90”), an immunophilin-like protein, XAP2, and the hsp90 interacting protein, p23. Ligand binding initiates a cascade of events that includes translocation to the nucleus, release of hsp90, and heterodimerization with ARNT. The ligand bound AhR-ARNT complex is capable of recognizing consensus sequences termed dioxin-response elements (“DRE”s) located in the promoter region of CYP1A1 and other responsive genes, thereby activating transcription. Known examples of AhR-associated proteins include, but are not limited to, hsp90 p23, XAP2, p60, hsp70, and p48.

The AhR protein contains several domains critical for function and is classified as a member of the basic helix-loop-helix/Per-Arnt-Sim (bHLH/PAS) family of transcription factors. The bHLH motif is located in the N-terminal of the protein. Members of the bHLH superfamily have two functionally distinctive and highly conserved domains. The first is the basic-region (b) which is involved in the binding of the transcription factor to DNA. The second is the helix-loop-helix (HLH) region which facilitates protein-protein interactions. Also contained with the AhR are two PAS domains, PAS-A and PAS-B, which are stretches of 200-350 amino acids that exhibit a high sequence homology to the protein domains that were originally found in the Drosophila genes period (Per) and single minded (Sim) and in AhR's dimerization partner, the aryl hydrocarbon receptor nuclear translocator (ARNT). The PAS domains support specific secondary interactions with other PAS domain containing proteins, as is the case with AhR and ARNT, so that heterozygous and homozygous protein complexes can form. The ligand binding site of AhR is contained within the PAS-B domain and contains several conserved residues critical for ligand binding. Finally, a Q-rich domain is located in the C-terminal region of the protein and is involved in co-activator recruitment and transactivation.

In colloboration with EPA TMO, Vincolozolin was identified as an AhR activator in HepG2 (hepatocyte reporter assay) (Wambaugh et al. 2014, Blystone et al. 2009). Further, it has been shown that AhR is essential for maintenance and survival of both intraepithelial lymphocytes (Li et al. 2011; Lee et al. 2012) and Group 3 innate lymphoid cells (Kiss et la. 2011,Qui et al. 2012). Distinct AhR ligands have been shown to promote Treg (TCDD, kyneurenine) or Th17 (FICZ) development (Quintana et al. 2008). AhR has also been shown to limit LPS endotoxin shock by promoting Tryptophan catabolism via IDO₁ (Bessede et al. 2014). Recently, it has been shown that AhR limits cord blood HSPC self-renewal (Rentas et al. 2016).

Accordingly, the term “aryl hydrocarbon receptor” or “AhR” as used herein refers to the 848 amino acid polypeptide having the amino acid sequence of:

(SEQ ID NO: 1)
MNSSSANITYASRKRRKPVQKTVKPIPAEGIKSNPSKRHRDRLNTELDRL
ASLLPFPQDVINKLDKLSVLRLSVSYLRAKSFFDVALKSSPTERNGGQDN
CRAANFREGLNLQEGEFLLQALNGFVLVVTTDALVFYASSTIQDYLGFQQ
SDVIHQSVYELIHTEDRAEFQRQLHWALNPSQCTESGQGIEEATGLPQTV
VCYNPDQIPPENSPLMERCFICRLRCLLDNSSGFLAMNFQGKLKYLHGQK
KKGKDGSILPPQLALFAIATPLQPPSILEIRTKNFIFRTKHKLDFTPIGC
DAKGRIVLGYTEAELCTRGSGYQFIHAADMLYCAESHIRMIKTGESGMIV
FRLLTKNNRWTWVQSNARLLYKNGRPDYIIVTQRPLTDEEGTEHLRKRNT
KLPFMFTTGEAVLYEATNPFPAIMDPLPLRTKNGTSGKDSATTSTLSKDS
LNPSSLLAAMMQQDESIYLYPASSTSSTAPFENNFFNESMNECRNWQDNT
APMGNDTILKHEQIDQPQDVNSFAGGHPGLFQDSKNSDLYSIMKNLGIDF
EDIRHMQNEKFFRNDFSGEVDFRDIDLTDEILTYVQDSLSKSPFIPSDYQ
QQQSLALNSSCMVQEHLHLEQQQQHHQKQVVVEPQQQLCQKMKHMQVNGM
FENWNSNQFVPFNCPQQDPQQYNVFTDLHGISQEFPYKSEMDSMPYTQNF
ISCNQPVLPQHSKCTELDYPMGSFEPSPYPTTSSLEDFVTCLQLPENQKH
GLNPQSAIITPQTCYAGAVSMYQCQPEPQHTHVGQMQYNPVLPGQQAFLN
KFQNGVLNETYPAELNNINNTQTTTHLQPLHHPSEARPFPDLTSSGFL,

as described by, e.g., NP_001612 or P35869, together with any naturally occurring allelic, splice variants, and processed forms thereof. Typically, AhR refers to human AhR. The term AhR is also used to refer to truncated forms or fragments of the AhR polypeptide, comprising, for example, specific AhR domains. Reference to any such forms of the AhR can be identified in the application, e.g., by “AhR (122-224).”

During canonical signaling, cytosolic AhR binds to a ligand, such as an OxC compound, which facilitates AhR translocation to the nucleus and eventually results in de novo transcription of target genes. The promoters of AhR target genes have the responsive element 5′-TNGCGTG-3′, termed “DRE” or “XRE” for “dioxin responsive elements” or “xenobiotic responsive elements.” The genes for xenobiotic-metabolizing enzymes (e.g., cytochrome P450) are well-known targets of AhR and are referred to herein as “AhR battery genes.” Hundreds of other genes also have DREs. Elucidation of the biochemistry of canonical AhR signaling has revealed several parameters that can fine-tune AhR activity. These include ligand characteristics, adapter molecules and transcriptional co-activators or co-repressors that regulate the extraordinary cell-specific activity of AhR.

Alternative pathways of AhR signaling have also been described. For instance, AhR can bind to retinoblastoma protein, estrogen receptor (ER), the transcription factor E2F1 and to the NFκB pathway subunits RelA and RelB. Evidence of AhR cross-talk with other signaling pathways, such as via kinases (src, JNK, p38, MAPK) or competition for transcription cofactors, has also been reported. AhR can act as a ubiquitin ligase, targeting the ER for proteasomal degradation. In these signaling pathways, AhR and the other proteins sometimes mutually repress each other's function. Indeed, bioinformatics analysis points to the existence of complex signal cross-talk between AhR and further transcription factors or transcription co-activators (C. Esser et al., Trends in Immunology 2009, Vol. 30: 9, pp. 447-454).

A number of low-molecular-weight chemicals qualify as endogenous or physiological “AhR ligands,” that is, they have binding dissociation constants (K_{d}) and effective concentrations at the level expected for a physiologically relevant AhR ligand. Physical fluid shear stress (which causes oxidation of low-density lipoproteins), the second messengers cAMP and Ca²⁺, serum and growth medium components all activate AhR responses (C. Esser et al., Trends in Immunology 2009, Vol. 30: 9, pp. 447-454). AhR has not yet been crystallized, so information on ligand-dependent structural changes is currently lacking. Ligand-protected protease digestion studies indicated that only one binding pocket for ligands exists (S. Kronenberg et al., Nucleic Acids Res. 28 (2000), pp. 2286-2291).

AhR ligands only need to meet minimal requirements for size and planar shape to fit into the AhR binding pocket. Consequently, a broad range of low-molecular-weight chemicals can activate AhR, albeit at different affinities ranging between 10⁻¹² and 10⁻³ M. Many ligands have two carbon ring systems, such as tryptophan derivatives, flavonoids and biphenyls. The AhR system is genetically polymorphic and different alleles influence responsiveness to AhR ligands (C. Esser et al., Trends in Immunology 2009, Vol. 30: 9, pp. 447-454). AhR ligands can generally be classified into two categories, synthetic or naturally occurring. The first ligands to be discovered were synthetic and members of the halogenated aromatic hydrocarbons (dibenzo-dioxins, dibenzofurans and biphenyls) and polycyclic aromatic hydrocarbons (3-methylcholanthrene, benzo(a)pyrene, benzanthracenes and benzoflavones).

Naturally occurring compounds that have been identified as ligands of AhR include derivatives of tryptophan such as indigo and indirubin, tetrapyroles such as bilirubinthe arachidonic acid metabolites lipoxin A4 and prostaglandin G, modified low-density lipoprotein and several dietary carotinoids.

Exemplary AhR ligands include, but are not limited to, endogenous ligands such as FICZ or 6-formylindolo[3,2-b]carbazole and 6,12-diformylindolo[3,2-b]carbazole or dFICZ (tryptophan photoproducts), bilirubin (product of heme metabolism by the liver), lipoxin A4 (eicosanoid with anti-inflammatory properties), ITE [2-(11-1-indole-3¹-carbonyl)-thiazole-4-carboxylic acid methyl ester] (isolated from lung tissues); Environmental pollutants (formed during combustion of organic material) such as 2,3,7,8-tetrachlorodibenzo-p-dioxin (TCDD), and Benz[a]pyrene; dietary ligands, such as quercetin (present in apples and onions), indol-3-carbinol (present in many Brassicaceae, e.g. cabbage) resveratrol (present in red wine), and curcumin (a spice); and drugs (synthetic), such as M50367 {3-[2-(2-phenylethyl) benzoimidazole-4-yl]-3-hydroxypropanoic acid, and VAF347 [4-(3-chloro-phenyl)-pyrimidin-2-yl]}.

Two of the most potent and well-characterized AhR antagonists include the synthetic flavonoid, 3′-methoxy-4′nitroflavone (“3M4NF”), and the indole derivative 3,3′-diindolylmethane (“DIM”). These compounds have been shown to function through direct competition for binding to the AhR ligand binding site (Henry et al., Mol. Pharmacol. 55:716-725 (1999); Hestermann et al., Mol. Cell. Biol 23:7920-7925 (2003)). The fate of the AhR upon binding of these structurally distinct antagonists is very different. Binding of 3M4NF to the AhR inhibits TCDD-mediated nuclear localization, ARNT dimerization, and DNA binding (Henry et al., Mol. Pharmacol. 55:716-725 (1999)). 3M4NF is believed to inhibit a conformational change within the AhR complex necessary for exposure of the nuclear localization sequence, resulting in retention of the AhR in the cytoplasmic compartment of the cell. Conversely, binding of DIM to the AhR allows nuclear localization, ARNT dimerization, and subsequent DNA binding. However, unlike the TCDD-bound AhR-ARNT dimer, this DIM-bound complex is incapable of recruiting the necessary co-factors responsible for initiating transcription (Hestermann et al., Mol. Cell. Biol 23:7920-7925 (2003)). Halogenated and nitro-substituted flavones can exhibit structure-dependent aryl hydrocarbon receptor (AhR) agonist and antagonist activities comparable to that observed for 2,3,7,8-tetrachlorodibenzo-p-dioxin (TCDD) (F. Lu et al., Biochemical Pharmacology. 51. 1077 (1996)).

As described herein, the X-ray crystal structure of Hif-2α PASB (PDBID:4GHI) was used to generate the homology model of human AhR-LBD using SWISSMODEL. ITE, oxazolone and TMO were built into this structure and docking orientation within the human AhR-LBD was determined by calculating the best energetically favorable scored solution by RosettaDock. The oxazole moiety of both oxazolone and TMO are coincident and indicate potential interactions with Histidine 291 (H₂₉₁), Serine 365 (S365), and Glutamine 383(Q383) residues of SEQ ID NO: 1. The phenyl ring of oxazolone can also facilitate potential aromatic (7t) interactions with phenylalanine 295 (F295) of SEQ ID NO: 1.

Accordingly, in some embodiments of the aspects described herein, the inhibitor of an OxC compound selectively binds to the OxC compound and inhibits or prevents binding of the OxC compound to the Aryl Hydrocarbon Receptor (AhR) of SEQ ID NO: 1. In further embodiments, the inhibitor of an OxC compound selectively binds to the OxC compound and inhibits or prevents binding of at one or more of H₂₉₁, F295, S365, and Q383 of SEQ ID NO: 1, thereby inhibiting AhR binding to an OxC compound.

In some embodiments of the aspects described herein, the inhibitor of an OxC compound is an Aryl Hydrocarbon Receptor (AhR) antagonist.

As used herein, an “AhR antagonist” refers to an inhibitor of an OxC compound, its metabolites, or a metabolic product induced by the OxC compound that does not provoke a biological response itself upon specifically binding to the AhR polypeptide or polynucleotide encoding the AhR, but blocks or dampens OxC compound-mediated responses, i.e., an AhR antagonist can bind but does not activate the AhR polypeptide or polynucleotide encoding the AhR, and the binding disrupts the interaction, or displaces an OxC compound, its metabolites, or a metabolic product induced by the OxC compound and/or inhibits the function of an OxC compound, its metabolites, or a metabolic product induced by the OxC compound binding to the AhR.

In some such embodiments, the AhR antagonist selectively binds the AhR at one or more amino acids selected from H₂₉₁, F295, S365, and Q383 of SEQ ID NO: 1, thereby inhibiting or preventing AhR binding to an OxC compound, its metabolites, or a metabolic product induced by the OxC compound.

In some embodiments of the aspects described herein, the inhibitor of an OxC compound is an antibody or antigen-binding fragment thereof that specifically binds to the OxC compound, its metabolites, or a metabolic product induced by the OxC compound.

In some such embodiments, the antibody or antigen-binding fragment thereof that specifically binds to the OxC compound inhibits or prevents binding of the OxC compound, its metabolites, or a metabolic product induced by the OxC compound to the AhR of SEQ ID NO: 1. In further embodiments, the antibody or antigen-binding fragment thereof inhibits or prevents binding of the OxC compound, its metabolites, or a metabolic product induced by the OxC compound to one or more amino acids selected from H₂₉₁, F295, S365, and Q383 of SEQ ID NO: 1, thereby inhibiting OxC compound binding to the AhR.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments can be produced by recombinant DNA techniques or by enzymatic or chemical cleavage of intact antibodies. Antigen-binding fragments include, inter alia, Fab, Fab′, F(ab′)₂, Fv, dAb, and complementarity determining region (CDR) fragments, single-chain antibodies (scFv), single domain antibodies, chimeric antibodies, diabodies, tetrabodies and other multimerized scFv moieties and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain) cleavage or via chemical cleavage of intact antibodies.

In some embodiments of the aspects described herein, the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)₂ fragments, a single chain antibody molecule, a diabody or a linear antibody.

The term “antigen-binding fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci . USA, 90:6444-6448 (1993)); (xi) “linear antibodies” comprising a pair of tandem Fd segments (VH-CH₁-VH-CH₁) which, together with complementary light chain polypeptides, form a pair of antigen binding regions (Zapata et al. Protein Eng. 8(10):1057-1062 (1995); and U.S. Pat. No. 5,641,870).

As described herein, an “antigen” is a molecule that is bound by a binding site on a polypeptide agent, such as an antibody or antibody fragment thereof. Typically, antigens are bound by antibody ligands and are capable of raising an antibody response in vivo. An antigen can be a polypeptide, protein, nucleic acid, lipid or other molecule. In the case of conventional antibodies and fragments thereof, the antibody binding site as defined by the variable loops (L1, L2, L3 and H₁, H₂, H₃) is capable of binding to the antigen. The term “antigenic determinant” refers to an epitope on the antigen recognized by an antigen-binding molecule, and more particularly, by the antigen-binding site of said molecule.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

In some embodiments of the aspects described herein, the antibody or antigen-binding fragment thereof that that specifically binds to the OxC compound is a monoclonal antibody. The term “monoclonal antibody,” as used herein, refers to an antibody that is part of a preparation of antibody molecules of single molecular composition. A population of monoclonal antibodies has a single binding specificity and affinity for a particular epitope, and the antibodies are identical except for possible naturally occurring mutations that can be present in minor amounts. Accordingly, the term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, 2^{nd} ed., (Cold Spring Harbor Laboratory Press, Cold Spring Harbor, 1988); Hammerling, et al. eds., in: Monoclonal Antibodies and T-Cell Hybridomas “In Research Monographs in Immunology, vol. 3 (J. L. Turk, General Editor) (Elsevier, N.Y., 1981), Kohler et al., Nature 256:495 (1975); can be made by recombinant DNA methods (see, e.g., U.S. Pat. No. 4,816,567); or can also be isolated from phage antibody libraries using the techniques described in Clackson et al., Nature 352:624-628 (1991) or Marks et al., J. Mol. Biol. 222:581-597 (1991), for example. The modifier “monoclonal” is not to be construed as requiring production of the antibody by any particular method.

In some embodiments of the aspects described herein, the antibody or antigen-binding fragment thereof that that specifically binds to the OxC compound is a humanized antibody. “Humanized” forms of non-human (e.g., murine) antibodies are chimeric antibodies that are engineered or designed to comprise minimal sequence derived from non-human immunoglobulin. Accordingly, a humanized antibody has one or more amino acid residues introduced into it from a source which is non-human. These non-human amino acid residues are often referred to as “import” residues, which are typically taken from an “import” variable domain. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. See Jones et al., 321 Nature 522 (1986); Riechmann et al., 332 Nature 323 (1988); Presta, 2 Curr. Op. Struct. Biol. 593 (1992).

In some embodiments of the aspects described herein, the antibody or antigen-binding fragment thereof that that specifically binds to the OxC compound is a composite human antibody. As used herein, a “composite human antibody” is a specific type of engineered or humanized antibody. Briefly, as used herein, “composite human antibodies” or “composite humanized antibodies” comprise multiple sequence segments (“composites”) derived from V-regions of unrelated human antibodies that are selected to maintain monoclonal antibody sequences critical for antigen binding of the starting precursor anti-human monoclonal antibody, and which have all been filtered for the presence of potential T-cell epitopes using “in silico tools” (Holgate & Baker, 2009). The close fit of human sequence segments with all sections of the starting antibody V regions and the elimination of CD4+ T cell epitopes prior to synthesis of the antibody allow this technology to circumvent immunogenicity in the development of ‘100% engineered composite human’ therapeutic antibodies while maintaining optimal affinity and specificity through the prior analysis of sequences necessary for antigen-specificity (Holgate & Baker, 2009).

In some embodiments of the aspects described herein, the antibody or antigen-binding fragment thereof that that specifically binds to the OxC compound is a non-engineered human antibody. A “human antibody,” “non-engineered human antibody,” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies. Vaughan et al., 14 Nature Biotechnol. 309 (1996); Sheets et al., 95 PNAS 6157 (1998); Hoogenboom & Winter, 227 J. Mol. Biol. 381 (1991); Marks et al., 222 J. Mol. Biol., 581 (1991).

Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. No. 5,545,807; U.S. Pat. No. 5,545,806; U.S. Pat. No. 5,569,825; U.S. Pat. No. 5,625,126; U.S. Pat. No. 5,633,425; U.S. Pat. No. 5,661,016; Marks et al., 10 Bio/Technology 779 (1992); Lonberg et al., 368 Nature 856 (1994); Morrison, 368 Nature 812 (1994); Fishwild et al., 14 Nat. Biotechnol. 845 (1996); Neuberger, 14 Nat. Biotechnol. 826 (1996); Lonberg & Huszar, 13 Intl. Rev. Immunol. 65 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies & Cancer Therapy 77 (Alan R. Liss, 1985); Boerner et al., 147 J. Immunol., 86 (1991); U.S. Pat. No. 5,750,373.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen Affinity matured antibodies are produced by procedures known in the art. Marks et al., 1992, describes affinity maturation by VH and VL domain shuffling. Random mutagenesis of CDR and/or framework residues is described by: Barbas et al., 91 PNAS 3809 (1994); Schier et al., 169 Gene 147 (1995); Yelton et al., 155 J. Immunol. 1994 (1995); Jackson et al., 154 J. Immunol. 3310 (1995); Hawkins et al., 226 J. Mol. Biol. 889 (1992).

In some embodiments of the aspects described herein, the OxC compound inhibitor is a small molecule. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

### Therapeutic Methods and Uses Comprising Inhibitors of OxC Compounds

As demonstrated herein, exposure to OxC compounds elicits an AhR-dependent pathway that can direct profound transcriptional changes in responsive tissues, such as the epithelium, that concomitantly influence barrier integrity at mucosal sites and modulate iNKT cell inflammatory responses, such as those associated with colitis. Thus, provided herein are therapeutic methods and uses comprising administering inhibitors of OxC compounds for the treatment of disorders associated with epithelial barrier integrity and iNKT cell inflammatory responses, such as colitis.

Accordingly, provided herein, in some aspects, are methods of treatment of a disease or disorder associated with epithelial cell barrier integrity and/or iNKT cells inflammatory responses, comprising administering to a subject in need thereof a therapeutically effective amount of a pharmaceutical composition comprising an inhibitor of an OxC compound and a pharmaceutically acceptable carrier.

As used herein, the phrase “a disease or disorder associated with epithelial cell barrier integrity” refers to any disease or disorder associated with or known to be caused, at least in part, by defects in epithelial cell barrier integrity or function. The methods described herein can be effective in preventing or treating the barrier dysfunction and attendant conditions and symptoms arising therefrom, associated with a variety of diseases and disorders, such as inflammation, atherosclerosis, and microbial pathogenesis. As particular nonlimiting examples, the conditions with which the epithelial cell barrier dysfunction occurs include inflammatory bowel disease, Crohn's disease, dermatitis, including allergic (contact dermatitis, such as irritant dermatitis, phototoxic dermatitis, allergic dermatitis, photoallergic dermatitis, contact urticaria, systemic contact-type dermatitis and the like and atopic dermatitis), as well as irritant dermatitis; gut-derived sepsis, a burn injury, a chemical contact injury, acute lung injury, asthma, COPD, neonatal necrotizing enterocolitis, severe neutropenia, toxic colitis, enteropathy, transplant rejection, pouchitis, pig-bel, uremic pericardial effusion, leakage in the vitreous of the eye, macular degeneration, retinal dysfunction, and infection (e.g., viral infection, bacterial infection, opportunistic bacterial infection, Clostridium dificile infection, Pseudomonas aeruginosa infection, Pseudomnonas-mediated ophthalmologic infection, Pseudomonas-mediated otologic infection and Pseudomonas-mediated cutaneous infection). An epithelium in the context of these aspects comprises at least two epithelial cells. In some embodiments, the epithelial cells are intestinal epithelial cells.

As used herein, the phrase “a disease or disorder associated with iNKT cells inflammatory responses” refers to any disease or disorder associated with or known to be caused, at least in part, by unwanted or excessive or increased iNKT cell immune responses, as the term is defined herein. Examples of diseases or disorders mediated by such iNKT cells include, but are not limited to, rheumatoid arthritis, systemic lupus erythematosus, type 1 diabetes, psoriasis, atherosclerosis, allergic asthma, graft versus host disease, haematological cancers.

As used herein, an “iNKT cell immune response” is a response by an iNKT cell to a stimulus, such as exposure to an OxC compound. Such responses by these cells can include, for example, cytotoxicity, proliferation, cytokine or chemokine production, and/or trafficking to a tissue site. In some embodiments of the compositions and methods described herein, an immune response being modulated is an inflammatory response.

As used herein, the terms “subject” or “individual” or “animal” or “patient” or “mammal,” refer to any subject, particularly a mammalian subject, preferably a human subject, for whom diagnosis, prognosis, or therapy is desired. In some embodiments, the subject has inflammatory bowel disease. In some embodiments, the subject had inflammatory bowel disease at some point in the subject's lifetime.

A subject can be one who has been previously diagnosed with or identified as suffering from or having a condition in need of treatment (e.g., colitis) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

In some embodiments of the aspects described herein, the disease or disorder associated with epithelial barrier integrity and/or iNKT cells inflammatory responses is an inflammatory bowel disease.

As used herein, the terms “colitides,” “colitis,” “inflammatory bowel disease,” and “IBD” refer to inflammatory conditions of the colon and/or small intestine, often characterized by abdominal pain, vomiting, diarrhea, rectal bleeding, cramps, and/or anemia. Examples of IBD include Crohn's disease, ulcerative colitis, and various classifications of colitides, e.g., idiopathic colitides (e.g., microscopic colitis, lymphocytic colitis, and collagenous colitis), iatrogenic colitides (e.g., including that associated with antibiotic administration, diversion colitis, neutropenic enterocolitis, disinfectant colitis, corrosive colitis, nonsteroidal anti-inflammatory drug and salicylate-induced colitis, toxic epidermal necrolysis, and other chemical-induced colitides), ischemic colitis, infectious colitides (e.g., Clostridium difficile colitis), eosinophilic colitis. In some embodiments, the condition (e.g., the IBD) is chronic, acute, and/or recurring. In some embodiments, the condition is colitis associated with antibiotic administration.

In some embodiments of the methods described herein, an inhibitor of an OxC compound is administered to a subject for treatment or management of a symptom of a flare-up of IBD (e.g., abdominal pain, rectal bleeding or blood in the subject's stool, loose bowel movements, loss of appetite, or abdominal inflammation). As used herein, a “flare” or “flare-up” of an inflammatory bowel disease (e.g., a colitis or Crohn's disease) refers to an acute aggravation of a symptom associated with the inflammatory bowel disease. Non-limiting examples of symptoms that, when increased in magnitude, are indicative of an IBD flare-up include, abdominal pain (e.g., pain that is unresponsive or less responsive to conventional management with, for example, pain medication or anti-spasmodic agents), increased rectal bleeding or blood in the subject's stool, an increase in the frequency of loose bowel movements, loss of appetite, and increased abdominal inflammation.

In some embodiments of the methods described herein, a subject being treated is first selected as having or at risk for having inflammatory bowel disease. Subjects having inflammatory bowel disease can be identified by a physician using current methods of diagnosing inflammatory bowel disease. Symptoms and/or complications of inflammatory bowel disease which characterize these conditions and aid in diagnosis are well known in the art and include but are not limited to, abdominal pain, loss of appetite, fatigue, bloody diarrhea, mucus in the stool, cramping, weight loss, and fever. Tests that may aid in a diagnosis of, e.g. inflammatory bowel disease include, but are not limited to, stool culture, abdominal CT or x-ray, or colonoscopy. A family history of inflammatory bowel disease, or exposure to risk factors for inflammatory bowel disease, such as the OxC compounds described herein, can also aid in determining if a subject is likely to have inflammatory bowel disease or in making a diagnosis of inflammatory bowel disease.

In some embodiments, the methods described herein include administration of an inhibitor of an OxC compound to a subject for the prophylactic treatment of IBD. In some embodiments, the methods described herein include administration of an inhibitor of an OxC compound to a subject with an increased risk of developing IBD (e.g., an increased familial risk, an increased genetic risk, or an increased environmental risk of developing one or more symptoms of IBD).

In some embodiments, the methods include administration of an inhibitor of an OxC compound to a subject who meets particular clinical criteria associated with an ineffective treatment or flare-up of an IBD. E.g., in some embodiments, an inhibitor of an OxC compound is administered to a subject found to have a particular clinical criterion associated with IBD. In some embodiments, administration of an inhibitor of an OxC compound is repeated until the subject falls below a particular criterion associated with IBD. In some embodiments, identification of the clinical criteria includes determination of a disease activity index for the particular subtype of inflammatory bowel disease.

In some embodiments of the methods described herein, the Crohn's Disease Activity Index (CDAI) is used to identify a clinical criterion associated with Crohn's disease. In some embodiments of the methods described herein, the Harvey-Bradshaw Index (HBI) is used to identify a clinical criterion associated with Crohn's disease. In some embodiments of the methods described herein, an inhibitor of an OxC compound is administered to a subject identified with an HBI score of at least 4. In some embodiments of the methods described herein, an inhibitor of an OxC compound is administered to a subject identified with an HBI score of at least 5. In some embodiments of the methods described herein, an inhibitor of an OxC compound is administered to a subject with a CDAI or HBI score that is higher than a baseline CDAI or HBI score for the patient, indicative of a remission or flare-up of the Crohn's disease (e.g., an HBI score that is at least 1 or 2 points higher than a baseline score for the subject).

In some embodiments of the methods described herein, a Mayo score is used to identify a clinical criterion associated with ulcerative colitis. In some embodiments of the methods described herein, a partial Mayo score is used to identify a clinical criterion associated with ulcerative colitis. In some embodiments of the methods described herein, an inhibitor of an OxC compound is administered to a subject with a Mayo score or partial May score that is higher than a baseline Mayo or partial Mayo, score for the subject (e.g., a Mayo score that is at least 1.5, 2.0, or 2.5 points higher than a baseline Mayo score for the subject; or a partial Mayo score that is at least 0.5, 1.0, or 1.5 points higher than a baseline partial Mayo score for the subject).

In some embodiments of the methods described herein, a pediatric ulcerative colitis activity index (PUCAI) is used to identify a clinical criterion associated with ulcerative colitis in pediatric subjects. In some embodiments of the methods described herein, an inhibitor of an OxC compound is administered to a pediatric subject identified with a PUCAI score of at least about 35, 40, 45, 50, 55, 60, 65, 70, or 75. In some embodiments, an inhibitor of an OxC compound is administered to a pediatric subject with a PUCAI score that is higher than a baseline PUCAI score for the pediatric subject (e.g., a PUCAI score that is at least 5, 10, or 15 points higher than a baseline PUCAI score for the pediatric subject).

In some embodiments, a measure of infection is used to identify a clinical criterion associated with an infectious colitides (e.g., Clostridium difficile colitis). In some embodiments, a total viable count (e.g., in the stool of the subject or in a sample from the bowel or intestine of the subject) of an infectious agent associated with the infectious colitis (e.g., Clostridium difficile) is the clinical criterion associated with the colitis. In some embodiments, a total spore count (e.g., in the stool of the subject or in a sample from the bowel or intestine of the subject) of an infectious agent associated with the infectious colitis (e.g., Clostridium difficile) is the clinical criterion associated with the colitis. In some embodiments, another marker of the presence of the infectious agent associated with the infectious colitis (e.g., a protein or nucleic acid specific to the infectious agent) is the clinical criterion associated with the colitis.

In some embodiments OxC compounds are present and causative of antibiotic colitis in the absence of Clostridum difficile and due to OxC compound generation by other microorganisms.

In other embodiments an individual has been iatrogenically exposed to a drug (e.g. a sulfa related compound) or to an environmentally derived OxC compound and has clinical symptoms derived from this exposure.

In some embodiments of the methods described herein, an inhibitor of an OxC compound is administered as a second line therapy or as part of a combinatorial treatment. For example, in some embodiments, an inhibitor of an OxC compound is administered to a subject who is partially or completely unresponsive to a first-line IBD treatment (e.g., an anti-inflammatory or immunosuppressant agent). In some embodiments, an inhibitor of an OxC compound is administered to a subject with a decreased chance of responding to a conventional IBD therapy (e.g., an anti-inflammatory or immunosuppressant agent). Non-limiting examples of other treatments can include anti-inflammatory (e.g. aminosalicylates or steroids), immunosuppressant agents, cyclosporine, and corticosteroids.

In some embodiments, the methods provided herein include co-administration of an inhibitor of an OxC compound with a conventional treatment for an inflammatory bowel disease, for example, an anti-inflammatory agent (e.g., a steroid, such as a corticosteroid steroid) or immunosuppressant. In some embodiments, the method includes co-administration of an inhibitor of an OxC compound with a conventional treatment for a flare-up of an inflammatory bowel disease, for example, an anti-inflammatory agent (e.g., a steroid, such as a corticosteroid steroid) or immunosuppressant.

In some embodiments, the methods provided herein include coadministration of an inhibitor of an OxC compound with a conventional treatment for ulcerative colitis, for example, an aminosalicylate, a corticosteroid, azathioprine, mercapopurine, cyclosporine, a TNF inhibitor (e.g., an anti-TNF-alpha monoclonal antibody, such as infliximab, adalimumab, or golimumab), or an integrin α₄β₇ inhibitor (e.g., an anti-integrin a4137 monoclonal antibody, such as vedolimumab).

In some embodiments, the methods provided herein include coadministration of an inhibitor of an OxC compound with a conventional treatment for Crohn's disease, for example, an aminosalicylate (e.g., sulfasalazine or mesalamine), a corticosteroid, azathioprine, mercapopurine, cyclosporine, a TNF inhibitor (e.g., an anti-TNF-alpha monoclonal antibody, such as infliximab, adalimumab, or certolizumab), an integrin α₄β₇ inhibitor (e.g., an anti-integrin α₄β₇ monoclonal antibody, such as vedolimumab), an integrin α₄β₃ inhibitor (e.g., an anti-integrin α₄β₇ monoclonal antibody, such as natalizumab), methotrexate, tacrolimus, an interleukin 12/interleukin 23 inhibitor (e.g., an anti-interleukin 12/interleukin 23 monoclonal antibody, such as ustekinumab), or an antibody (e.g., metronidazole or ciprofloxacin).

In some embodiments of the aspects described herein, the pharmaceutical composition comprising an inhibitor of an OxC compound and a pharmaceutically acceptable carrier inhibits or reduces or alleviates one or more symptoms associated with the disorder.

By “reduce” or “inhibit” in terms of the treatment methods described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom. “Complete inhibition” is a 100% inhibition as compared to a reference level. A decrease can be preferably down to a level accepted as within the range of normal for an individual without a given disorder. Reduce or inhibit can refer to, for example, the symptoms of the disorder being treated. For example, in the case of colitis, such symptoms to be reduced or inhibited include, but are not limited to: increased weight loss, colon shortening, histopathology (based on blind scoring by a pathologist), and/or expression of one or more cytokines characteristic of colitis.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as IBD. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “effective amount” as used herein refers to the amount of an inhibitor of an OxC compound needed to alleviate at least one or more symptom of the disease or disorder, and relates to a sufficient amount of pharmacological composition to provide the desired effect, for example, treating IBD. The term “therapeutically effective amount” therefore refers to an amount of an inhibitor of an OxC compound using the methods as disclosed herein, that is sufficient to effect a particular effect when administered to a typical subject. An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the inhibitor of an OxC compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The agents useful according to the compositions and methods described herein, including antibodies and other polypeptides, are isolated agents, meaning that the agents are substantially pure and are essentially free of other substances with which they may be found in nature or in vivo systems to an extent practical and appropriate for their intended use. In particular, the agents are sufficiently pure and are sufficiently free from other biological constituents of their host cells so as to be useful in, for example, producing pharmaceutical preparations. Because an isolated agent can be admixed with a pharmaceutically acceptable carrier in a pharmaceutical preparation, the agents may comprise only a small percentage by weight of the preparation.

The inhibitors of an OxC compound described herein can be administered to a subject in need thereof by any appropriate route which results in an effective treatment in the subject. As used herein, the terms “administering,” and “introducing” are used interchangeably and refer to the placement of an agent, such as the inhibitor of an OxC compound, into a subject by a method or route which results in at least partial localization of such agents at a desired site, such as an epithelial cell or site of inflammation, such that a desired effect(s) is produced.

In some embodiments, the inhibitors of an OxC compound described herein are administered to a subject by any mode of administration that delivers the agent systemically or to a desired surface or target, and can include, but is not limited to, injection, infusion, instillation, and inhalation administration. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the inhibitors of an OxC compound for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of an inhibitor of an OxC compound other than directly into a target site, tissue, or organ, such that it enters the subject's circulatory system and, thus, is subject to metabolism and other like processes.

For the clinical use of the methods described herein, administration of an inhibitor of an OxC compound can include formulation into pharmaceutical compositions or pharmaceutical formulations for parenteral administration, e.g., intravenous; mucosal, e.g., intranasal; ocular, or other mode of administration. In some embodiments, an inhibitor of an OxC compound can be administered along with any pharmaceutically acceptable carrier compound, material, or composition which results in an effective treatment in the subject. Thus, a pharmaceutical formulation for use in the methods described herein can contain an inhibitor of an OxC compound in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an inhibitor of an OxC compound. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) pH buffered solutions; (19) polyesters, polycarbonates and/or polyanhydrides; (20) bulking agents, such as polypeptides and amino acids (21) serum components, such as serum albumin, HDL and LDL; (22) C2-C12 alchols, such as ethanol; and (23) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The inhibitors of an OxC compound described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, the inhibitors of an OxC compound can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Further embodiments of the formulations and modes of administration of inhibitors of an OxC compound that can be used in the methods described herein are illustrated below.

Parenteral Dosage Forms. Parenteral dosage forms of inhibitors of an OxC compound can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. Inhibitors of an OxC compound can be packaged in a pressurized aerosol container together with suitable propellants, for example, hydrocarbon propellants like propane, butane, or isobutane with conventional adjuvants. Inhibitors of an OxC compound can also be administered in a non-pressurized form such as in a nebulizer or atomizer. An inhibitor of an OxC compound can also be administered directly to the airways in the form of a dry powder, for example, by use of an inhaler.

Suitable powder compositions include, by way of illustration, powdered preparations of an inhibitor of an OxC compound thoroughly intermixed with lactose, or other inert powders acceptable for intrabronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the inhibitors of an OxC compound described herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the methods described herein, inhibitors of an OxC compound can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound's onset of action, duration of action, plasma levels within the therapeutic window, and peak blood levels. In particular, controlled- or extended-release dosage forms or formulations can be used to ensure that the maximum effectiveness of an inhibitor of an OxC compound is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the inhibitors of an OxC compound described herein. Examples include, but are not limited to, those described in U.S. Pat. Nos. 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

In some embodiments, an inhibitor of an OxC compound for use in the methods described herein is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the inhibitor of an OxC compound administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Diagnostics Methods and Uses Comprising OxC Compounds

Provided herein, in some aspects, are detection methods, such as PCR-based methods, for targeted detection for the presence of OxC compounds, such asTOMMs, in biological samples from a subject, such as feces of patients with IBD (or inflammatory disorders in other body sites such as the skin) as a diagnostic and/or predictive measure of disease onset. Such diagnostic assays and methods can further comprise, in some embodiments, treatment steps using an OxC compound inhibitor, as described herein.

Accordingly, provided herein in some aspects are assays and methods for detecting the presence of an OxC compound in a biological sample comprising measuring a level of an OxC compound in a biological sample obtained from a subject, wherein if the level of the OxC compound is relative to a control sample, the sample is identified as containing a an OxC compound.

In some aspects, provided herein are assays and methods for detecting the presence of a thiazole/oxazole-modified microcin (TOMM) in a biological sample comprising measuring a level of a TOMM in a biological sample obtained from a subject, wherein if the level of a TOMM is increased at least 1.5 fold relative to a control sample, the sample is identified as containing a TOMM. The terms “increased,” “increase” or “enhance” in connection with the level or amount of an OxC compound, such as a TOMM, in a biological sample obtained from a subject are all used herein to generally mean an increase by a statically significant amount. For the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference value or level, or at least about a 1.5-fold, at least about a 1.6-fold, at least about a 1.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level or control sample. In some embodiments, an increase is at least one standard deviation greater than, or at least two standard deviations, or more, greater than a median or mean reference level or a level found in a control sample. Such median or mean reference levels can be obtained, for example, from five or more samples obtained from subjects not having IBD, or from five or more samples obtained from the same subject at different timepoints.

The reference level can obtained or measured in a reference biological sample, such as a reference sample obtained from an age-matched normal control (e.g., an age-matched subject not having IBD), or a reference sample from the same subject at an earlier timpoint, for example, a “first biological sample.” A “reference value” is thus, in some embodiments, a predetermined reference level, such as an average or median amount or level of an OxC compound, such as a TOMM, obtained from, for example, biological samples from a population of healthy subjects that are in the chronological age group matched with the chronological age of the tested subject.

As used herein, a “sample” or “biological sample” or “test sample” can refer to a solid, semi-solid or liquid sample, including, but not limited to, fecal samples, urine, sputum, a tissue sample, such as skin sample, a cellular sample, a cellular extract, plasma, serum, blood, cord blood, body secretions from the nose, oropharynx, gastrointestinal tract, bile or genitourinary tract, tissue biopsies of any organ, a tissue fluid such as cerebrospinal, occular or joint fluids, or any combination thereof. A sample can be obtained by removing a sample from a subject, but can also be accomplished by using a previously isolated sample (e.g. isolated at a prior timepoint and isolated by the same or another person).

In some embodiments of these assays and methods, the biological sample is a fecal, sputum, urine, or skin sample.

In some embodiments, the sample can be an untreated sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein comprise a step of obtaining a biological sample from the subject. In some embodiments, the subject can be a human subject. In some embodiments, the subject can be a subject in need of treatment for (e.g. having or diagnosed as having) IBD or a subject at risk of or at increased risk of developing a disease or disorder, such as IBD as described elsewhere herein. In some embodiments, the subject can be a subject determined to have one or more genetic variations known to be associated with a disease or disorder, such as IBD.

In some embodiments of the methods, assays, and systems described herein, the measuring of the level of a TOMM in a sample comprises mass spectrometry, PCR, or an immunoassay.

As used herein, “determining the amount of an OxC compound,” or “measuring or quantifying the amount of of an OxC compound,” refers to any investigative or analytic method, procedure, or assay that can be used to for qualitatively assessing or quantitatively measuring the presence or amount of one or more OxC compounds, such as TOMMs, in a biological sample. For the aspects described herein, “determining,” “measuring,” “quantifying,” or “assaying” encompasses any number of techniques and methods and includes, but is not limited to, mass spectrometry; methods of “quantitative” and “semi-quantitative” amplification, including fluorogenic quantitative PCR, ligase chain reaction (LCR), transcription amplification, self-sustained sequence replication, dot PCR, and linker adapter PCR, etc.; immunoassay techniques, including sandwich enzyme-linked immunoassays (ELISA), immunoabsorbent assays, immunoprecipitation assays, immunoblotting assays, radioimmunoassays (RIA), competitive binding assays, homogeneous assays, heterogeneous assays, etc.

As described herein, in some embodiments, OxC compounds, such as TOMMs, can be detected and quantified by mass spectrometry (MS). Exemplary mass spectrometric methods include, but are not limited to, time of flight (TOF), quadrupole, triple quadrupole, high resolution and other mass spectrometric methods. Additionally, exemplary ionization methods, prior to mass spectrometric analysis, include but are not limited to matrix assisted laser desorption ionization (MALDI) and electro-spray ionization (ESI). OxC compounds can be purified prior to injection into the mass spectrometer using liquid chromatography (LC) or gas chromatography (GC). In some embodiments, OxC compounds can be detected and quantified using LC-MS, GC-MS, or MALDI-TOF mass spectrometry.

In some embodiments of the assays described herein, if the sample is identified as containing a TOMM, the assay further comprises the step of administering an inhibitor of an OxC compound to the subject from whom the biological sample was obtained. Such administration steps can be performed using any of the treatment methods described herein.

Some embodiments of the technology described herein can be defined according to any of the following numbered paragraphs:
1. A pharmaceutical composition comprising an inhibitor of an Oxazole containing (OxC) compound and a pharmaceutically acceptable carrier, wherein the OxC compound is a compound of any of Formula I

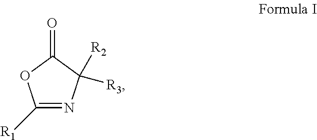

### Formula II

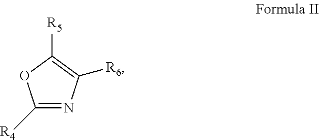

### Formula III

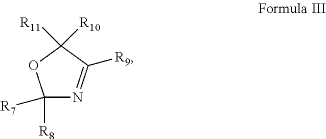

### or Formula IV

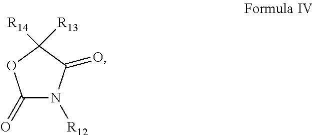

wherein R₁-R₁₄ are each independently selected from the group consisting of hydrogen, alkyl, alkenyl, alkynyl, aryl, heteroaryl, alkoxy, amino, and carbonyl, provided that each of Formulas I-IV has two or more R groups which are not hydrogen.
2. The pharmaceutical composition of paragraph 1, wherein the compound of Formula I is Oxazolone

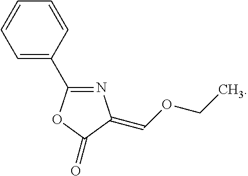 [SMILES: CCO/C=C1\N=C(c2ccccc2)OC1=O]

3. The pharmaceutical composition of paragraph 1, wherein the compound of Formula II is selected from

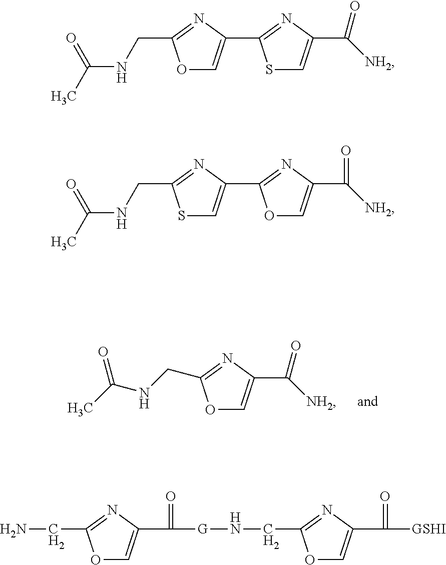

“GSHI” is disclosed as SEQ ID NO: 2.
4. The pharmaceutical composition of paragraph 1, wherein the compound of Formula III is 2,4,5 -trimethyl-2,5 -dihydro -1,3 -oxazole (TMO):

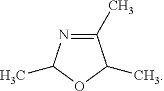

5. The pharmaceutical composition of paragraph 1, wherein the compound of Formula IV is vinclozolin:

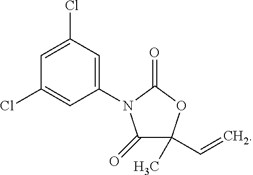 [SMILES: C=CC1(C)OC(=O)N(c2cc(Cl)cc(Cl)c2)C1=O]

6. The pharmaceutical composition of paragraph 1, wherein the OxC compound of

Formula II is a thiazole/oxazole-modified microcin (TOMM).
7. The pharmaceutical composition of paragraph 6, wherein the TOMM is microcin B17 or mutants or fragments thereof
8. The pharmaceutical composition of any one of paragraphs 1-7, wherein the inhibitor of an OxC compound specifically binds to the OxC compound, its metabolites, or a metabolic product induced by the OxC compound.
9. The pharmaceutical composition of paragraph 8, wherein the inhibitor of an OxC compound specifically binds to the OxC compound and inhibits or prevents binding of the OxC compound, its metabolites, or a metabolic product induced by an OxC compound to the Aryl Hydrocarbon Receptor (AhR) of SEQ ID NO: 1 and its activation.
10. The pharmaceutical composition of paragraph 9, wherein the inhibitor of an OxC compound inhibits or prevents binding of the OxC compound, its metabolites, or a metabolic product induced by an OxC compound to one or more amino acids selected from H₂₉₁, F295, S365, and Q383, thereby inhibiting AhR binding to an OxC compound.
11. The pharmaceutical composition of any one of paragraphs 1-7, wherein the inhibitor of an OxC compound is an Aryl Hydrocarbon Receptor (AhR) antagonist.
12. The pharmaceutical composition of paragraph 11, wherein the AhR antagonist binds to the Aryl Hydrocarbon Receptor (AhR) of SEQ ID NO: 1 at one or more amino acids selected from H₂₉₁, F295, S365, and Q383 of SEQ ID NO: 1, and inhibits or prevents AhR binding to an OxC compound, its metabolites, or a metabolic product induced by an OxC compound.
13. The pharmaceutical composition of any one of paragraphs 1-12, wherein the inhibitor of an OxC compound is an antibody or antigen-binding fragment thereof
14. The pharmaceutical composition of paragraph 13, wherein the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)₂ fragments, a single chain antibody molecule, a diabody or a linear antibody.the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)₂ fragments, a single chain antibody molecule, a diabody or a linear antibody.
15. The pharmaceutical composition of any one of paragraphs 1-12, wherein the inhibitor of an OxC compound is a small molecule.
16. A method of treatment of a disease or disorder associated with epithelial barrier integrity and/or iNKT cell-mediated inflammatory responses, comprising administering to a subject in need thereof a therapeutically effective amount of a pharmaceutical composition of any one of paragraphs 1-15.
17. The method of paragraph 16, wherein the disease or disorder associated with epithelial barrier integrity and/or iNKT cell-mediated inflammatory responses is an inflammatory bowel disease.
18. The method of paragraph 17, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.
19. An assay for detecting the presence of a thiazole/oxazole-modified microcin (TOMM) in a biological sample comprising measuring a level of a TOMM in a biological sample obtained from a subject, wherein if the level of a TOMM is increased at least 1.5 fold relative to a control sample, the biological sample is identified as containing a TOMM.
20. The assay of paragraph 19, wherein the biological sample is a fecal or skin sample.
21. The assay of any one of paragraphs 19-20, further comprising a step of obtaining the biological sample from the subject.
22. The assay of any one of paragraphs 19-21, wherein the biological sample is obtained from a subject in need of treatment for IBD, or a subject at risk of or at increased risk of developing IBD.
23. The assay of any one of paragraphs 19-22, wherein the measuring of the level of a

TOMM in the biological sample comprises mass spectrometry, PCR, or an immunoassay.
24. The assay of any one of paragraphs 19-23, wherein if the biological sample is identified as containing a TOMM, the assay further comprises the step of administering a pharmaceutical composition of any one of paragraphs 1-15.

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 Dec); and Riechmann et al., Reshaping human antibodies for therapy, Nature 1988 Mar 24, 332(6162):323-7.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Inflammatory bowel disease (IBD) is a complex disorder, influenced by environmental and microbial factors in a genetically susceptible host, which results in chronic relapsing and remitting inflammation of the gastrointestinal tract. Epidemiological studies have emphasized the importance of environmental elements, such as diet, as major risk factors for disease pathogenesis, [5-7] yet the identification and structural characterization of these natural and synthetic environmental factors that influence colitis development and the mechanisms by which they do so were previously unknown.

Recent work has established a model of mucosal homeostasis in which distinct, tissue-specific responses by CD1d, through direct crosslinking or through presentation of self, microbial, and environmentally derived lipid antigens to natural killer T (NKT) cells, serves to maintain epithelial barrier integrity[10-12] or promote intestinal inflammation, respectively. [8-11]These insights, which model the development of ulcerative colitis, have largely been established utilizing a model of oxazolone-induced colitis which is characterized by massive bowel wall edema and dense infiltration of the superficial mucosal layers with polymorphonuclear granulocytes together with ulceration of the epithelial cell layer [10,12]. It has been generally hypothesized that oxazolone functions as an immune hapten, which modifies self or microbial lipids conferring agonistic properties that stimulate CD1d-restricted NKT cells.

However, we questioned whether oxazolone is representative of a larger group of environmental and/or microbial factors that induce colitis in a host harboring an NKT cell pathway made susceptible to immune triggering either due to improper microbial induced immune education (as in early life [9] and/or in a genetically susceptible host). Consistent with this, we have recognized that oxazolone is a highly reactive compound featuring a heterocyclic oxazole ring.[13,14]. We have also observed that oxazole-containing compounds are highly abundant and enriched in microbial secretion systems, [15-21] diet,[22-27] and widely used in industrial applications (i.e. fungicides) that can be ingested.[14,28].

Without wishing to be bound or limited by theory, we hypothesized that common structural elements of these compounds define a class of environmental stimuli that trigger defects in epithelial barrier protection, thereby promoting the development of colitis through a pathway that is derived from CD1d-restricted NKT cells. As described herein, our computational models indicate that oxazole containing compounds (OxC compounds) are a novel class of molecules recognized by a ligand activated transcription factor, aryl hydrocarbon receptor (AhR) an important sensor of polycyclic aromatic hydrocarbons, that mediates that toxic effects of environmental xenobiotics, but can also direct development and function of specific immune subsets involved in mucosal immunity and autoimmune disease. The experimental evidence described herein also indicates that OxC compounds elicit cellular responses that modulate specific epithelial transcriptional targets and influence NKT cell immunomodulatory function, in an AhR dependent manner, providing a mechanistic link between exposure to “oxazolone-like” chemicals in the environment to epithelial barrier defects, mucosal dysbiosis and intestinal inflammation observed in IBD.

Genome-wide association studies have identified >150 risk loci involved in the development of the two major forms of IBD, Crohn's disease and ulcerative colitis, some of which have been validated in experimental models [45,46]. Additionally, IBD has been characterized by microbial dysbiosis and studies have begun to functionally define the microbiota involved in IBD development and pathogenesis.[8,9,39,47]

However, similar achievements defining the causative environmental agents of IBD have lagged, despite compelling epidemiological evidence linking lifestyle and disease prevalence.[1] Indeed the rapid global increases in IBD implicate a critical role for yet-to-be defined environmental forces underlying this phenomenon. Chemically induced mouse models of intestinal inflammation, like oxazolone-induced colitis have been crucial experimental tools in investigating the pathophysiology and testing of therapeutic strategies for IBD.[10-12,49]The model phenotypically resembles UC and is mediated primarily by interleukin-4 (IL-4) and -13 (IL-13) producing NKT cells in T helper (Th2) prone strains of animals such as SJL/J.[10-1 2] NKT cells recognize lipid antigens when presented by CD1d molecules on IECs and APCs. [52-54] NKT cells are classified into two groups: group 1 NKT cells express a canonical invariant (i) T cell receptor (TCR)-α chain associated with a limited repertoire of TCR-β chains (iNKT); group 2 NKT cells express a diverse (a array of TCR-α and -β chains (dNKT).[54-57] iNKT recruitment to mucosal tissues, including the gut, is mediated in part by microbial regulated CXCL16 from both parenchymal and hematopoietic sources in the colon.[9]Subsequently, iNKT activation occurs via lipid antigen sensing on CD1d-bearing resident mucosal APCs. Lipidation of CD1d molecules is carried out by microsomal triglyceride transfer protein (MTP), an endoplasmic reticulum resident protein also involved in CD1d biogenesis.[8, 41]Genetic and pharmacological depletion or inactivation of iNKT cell populations, using anti-CD1d or anti-NK1.1 and animals with targeted deletions of CD1d or the invariant TCR Ja18 chain, respectively,[8-12, 57-59]have demonstrated an essential role for iNKT activity in the development of oxazolone-induced colitis. Significantly, we measured elevated CD1d restricted IL-13 production from lamina propria T cells from UC patients, compared to Crohn's patients or patients with non-inflammatory malignant disease, indicating NKT cells as a candidate source of inflammation and potential etiological factor, and supporting oxazolone colitis as a model to study epithelial barrier function and NKT biology in the context of inflammation of the distal colon.[11, 96]

Recent studies from our laboratory have revealed a distinct role for CD1d activity in intestinal epithelial cells (IEC) that promote epithelial barrier activity through the production of interleukin 10 (IL-10).[8]Crosslinking CD1d on IECs leads to STAT3 activation and induction of transcriptional targets: IL-10 and heat shock protein 110 (HSP110) that attenuate mucosal inflammation after oxazolone treatment. Epithelial specific deletion of MTP or CD1d results in decreased colonic CD1d, IL-10 and HSP110 protein expression and consequently increased sensitivity to oxazolone challenge. [8] Thus, tissue-specific MTP and CD1d from IECs or hematopoietic sources promote barrier protective pathways or iNKT cell dependent intestinal inflammation, respectively.

Furthermore, it is understood that mucosal dysbiosis is a major component of colitis progression, germ free (GF) animals harbor elevated levels of NKT cells in colonic lamina propria and thus are more sensitive to oxazole administration compared to mice raised in conventional environments (SPF).[9,39]These studies demonstrate how microbial-derived signals generated in part during neonatal life can influence host sensitivity to environmental factors, such as exposure to oxazolone, which trigger exaggerated iNKT cell responses in mucosal tissue.

These observations led us to determine whether oxazolone is representative of a broader system of environmental factors with shared sub-structure in the pathogenesis of colitis. During the course of this analysis, we determined that the oxazole 5 membered ring structure, first identified by Erlenmeyer, features oxygen and nitrogen at the 1- and 3-positions following Hantzsch-Widman nomenclature and is closely related to the 0-lactam ring systems found in penicillin [FIG. 1a].[61] Oxazole rings are an abundant structural feature within the class heteroaromatics, which some estimate to make up about one half of all known compounds.[62, 97]The oxazole ring contains numerous reactive sites allowing for a wide variety of transformations, making it a popular industrial substrate for synthetic production of heterocyclic compounds[64], including amino acid synthesis.[61,65]0xazole-ring-containing (OxC) compounds are also naturally abundant in diet as a component of many food systems including coffee, cocoa, barley, potato, and meat products[65]and have been appropriated as an additive to fruits and vegetables as a pesticide. For instance 55,000 pounds of vinclozolin[FIG. 1b] an OxC compound with anti-fungal properties, was used to treat crops annually in the US, according to recent estimates.[28]The microbial world is also an abundant source of oxazoles as part of a structurally diverse class of ribosomally derived peptides dubbed thiazole/oxazole-modified microcins (TOMMs) generated through posttranslation installation of heterocycles derived from cysteine, serine, and threonine residues and widely disseminated across the phylogenetic spectra of bacterial secretion systems, including commensals such as Proteobacteria associated with IBD pathogenesis.[15-21,98,99]TOMM products are functionally diverse with metabolic, inflammatory, and anti-microbial properties and thus selectively endow ecological advantages. For instance, many Escherichia coli (E. coli) strains encode an 7 gene operon (mcba-g) encoding the scaffold and enzymatic machinery needed to produce the TOMM B17 [FIG. 7a], an oxazole-ring containing heterocyclic peptide that inactivates bacterial DNA gyrases.[18] B17 competent strains secrete B17 toxin that targets susceptible sub-populations lacking the immunity gene (mcbg) encoded within the operon.[15]Though precise measurements of the human rate of exposure to OxC compounds are lacking, heuristic methods to evaluate human exposure to a select group of OxC compounds found in urine samples estimate exposures of in the 10⁻⁷g/kg of body weight per day.[100]

Defining the structural features underlying the colitogenic potential of oxazole containing dietary, microbial, and environmental compounds.

The oxazolone-induced model of colitis faithfully reproduces many of the pathological features of UC[10-12]and has been widely used to understand the development of acute and chronic colonic inflammation caused by epithelial barrier disruption and exaggerated mucosal immune responses.[8,9,11,39] These studies have emphasized that interactions between CD1d-bearing intestinal epithelial cells (IEC), myeloid antigen presentation cells (APC), and NKT cells modulate mucosal integrity under homeostatic and disease contexts. Our studies, as described herein, have identified structural features of oxazole-containing (OxC) compounds from a variety of sources (i.e. dietary additives, fungicides, microbial metabolites) that mediate expression of key genes involved in epithelial barrier protection and can influence NKT cell responses to lipid antigens in vitro.

As described herein, we have devised an empirical approach to resolve the colitogenic potential of the OxC family of compounds in vivo by systematically substituting key positions within the 5-membered ring structure and evaluating epithelial function, immune response, and histological pathology associated with the model of ulcerative colitis induced through oxazolone administration. In this manner, the minimal structural unit responsible for colitogenic activity by OxC compounds derived from the environment, diet and microbiota is defined. Cell specific responses are evaluated by transcriptional profiling of CD1d bearing IECs, myeloid derived APC subsets, and NKT cells in response to a panel of OxC compounds (including oxazolone itself) to identify gene targets responsible for mediating the effects of oxazolone/OxC compounds on epithelial barrier function and subsequent inflammatory response. Finally, these findings are used to develop a novel model of colitis triggered by microbial-derived OxC compounds by exploiting a candidate pathobiont engineered to express B17 microcin, an OxC compound that modulates CD1d-restricted iNKT responses. These studies support a mechanism in which OxC compounds represent an important class of environmental triggers of intestinal inflammation through direct functional effects on the intestinal epithelium and other CD1d-bearing APCs by triggering NKT responses in a manner that results in intestinal inflammation of the distal colon.

Identifying and characterizing molecular sensors of colitogenic oxazoles containing compounds.

Heterocyclic oxazoles are a chemically reactive component of oxazolone and a prevalent structural feature in microbial derived secretion systems, diet, antibiotics, herbicides and synthetic chemicals.[15-28] Mammalian models of oxazolone-induced colitis have demonstrated the colitogenic potential of these compounds in rodent models of intestinal inflammation[10,12,41] but the primary molecular events responsible for sensing and transducing oxazolone inflammatory stimuli are unknown.

The data provided herein indicate that specific changes in cellular function occur in response to a minimal oxazole-containing structure leading to modulation of signature NKT associated targets. These include microsomal triglyceride transfer protein (Mttp), which our group has shown directly lipidates CD1d, controlling its antigen presentation activity and barrier function in epithelial cells[41,42] and Cxcl16, which functions as a chemoattractant for NKT cells via CXCR6 binding.[9,43]

As described herein, computational analyses of oxazolone substructure indicate that oxazole containing compounds are a novel class of ligands recognized by the aryl hydrocarbon receptor (AhR), a critical sensor for environmentally derived aromatic toxins and an important modulator immune cell development and effector function.[30-38] As shown herein, we have further validated that oxazolone and OxC compounds induce transcriptional responses in an AhR-dependent manner. Herein, we evaluate and characterize the molecular interactions between OxC compounds and AhR and decipher a role for AhR in regulating transcriptional responses mediating epithelial barrier protection and NKT cell responses, both of which contribute to oxazolone induced inflammation and ulcerative colitis.

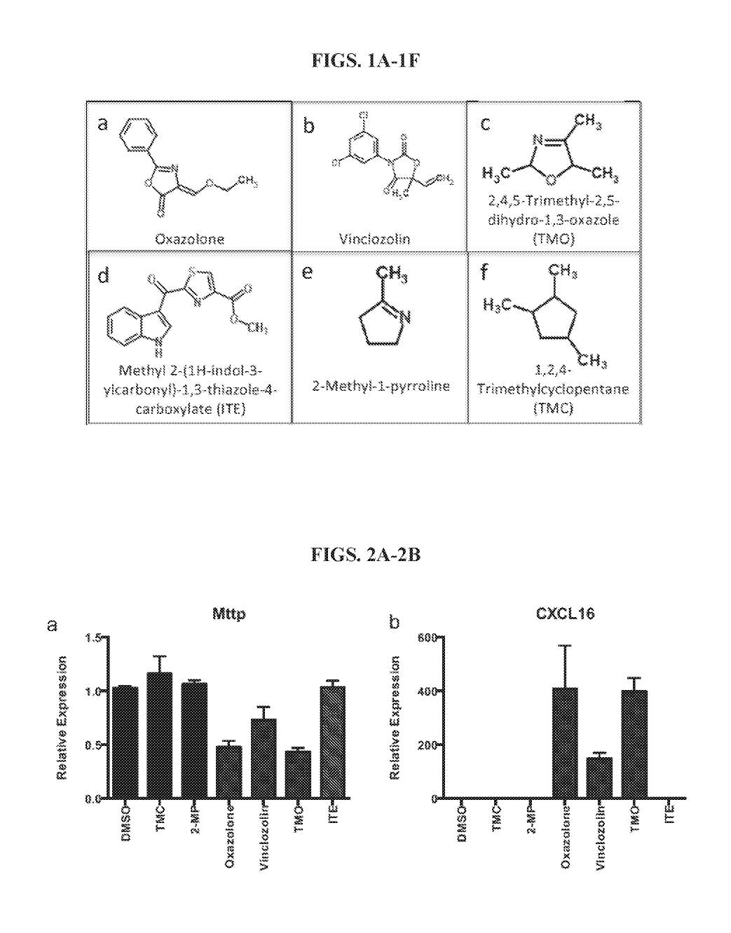

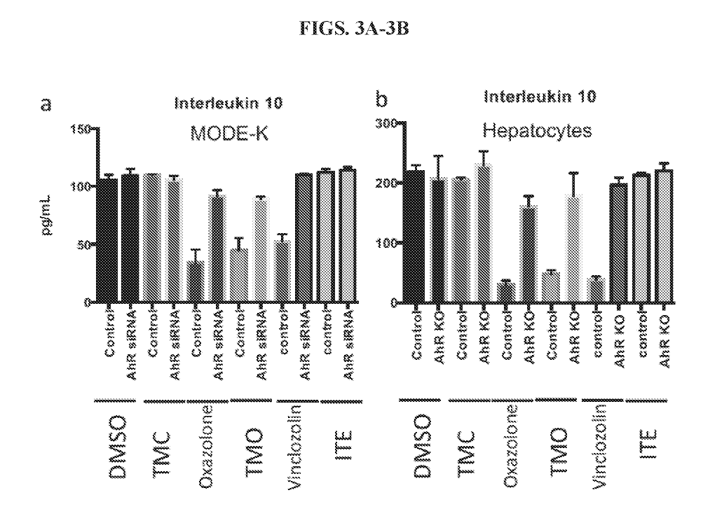

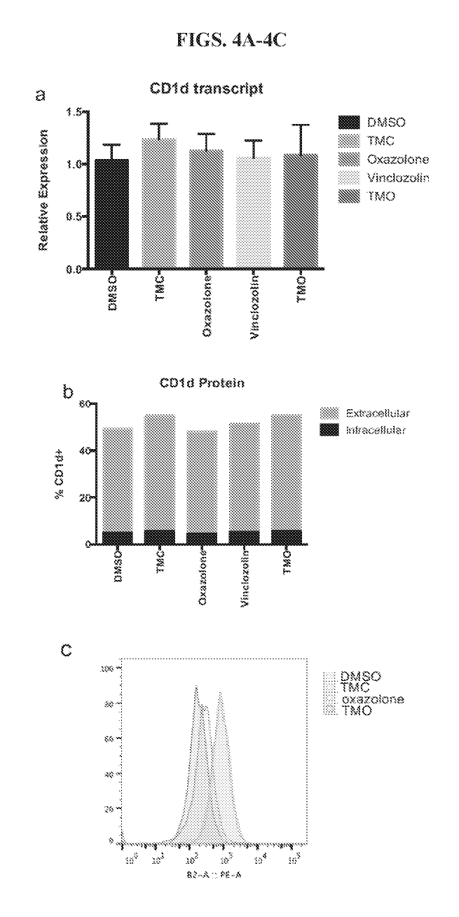

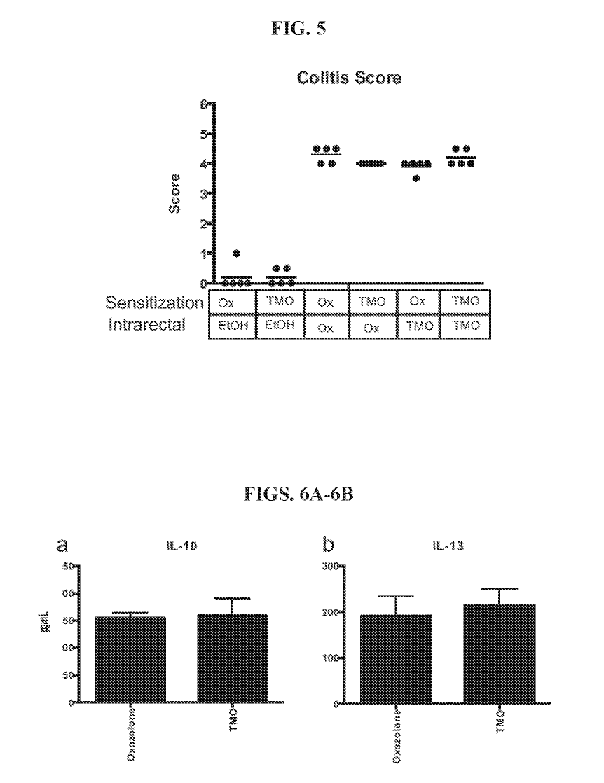

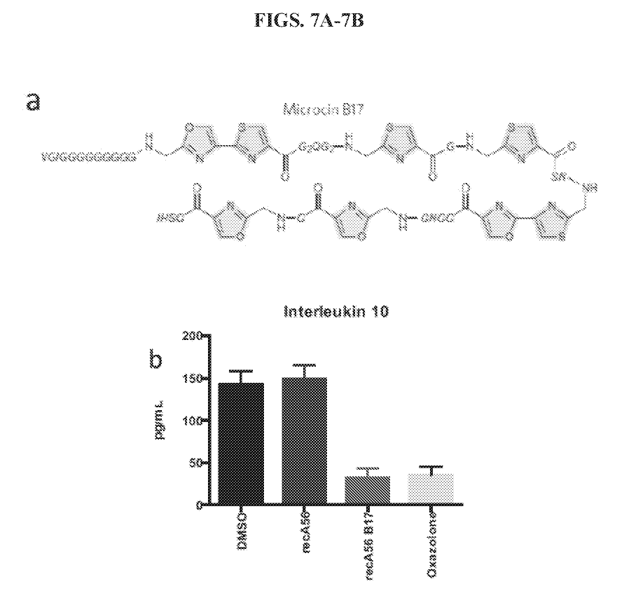

Our prototype compound 4-ethoxymethylene-2-phenyl-2-oxazol-5-one (oxazolone) possesses colitogenic potential through a mechanism dependent upon CD1d and NKT cells.[10,12] Classically, oxazolone has been proposed to act as a haptenating agent based on the detection of immunoglobulins directed against oxazolone after intrarectal administration,[9] indicating that re-exposure to this “antigen” triggers an inflammatory responses; however no direct evidence of oxazolone-peptide or —lipid complexes in colonic mucosa has been detected. Stimulation of MODE-K (epithelial) cells leads to distinct positive and negative transcriptional outcomes for Mttp and Cxcl16 [FIG. 2], two genes previously implicated in maintaining epithelial barrier activity and promoting iNKT recruitment to the colon, respectively, and involved in the pathogenesis of oxazolone colitis. [8,9] Thus, we found that oxazolone elicits cell intrinsic responses and modulates specific gene targets involved in NKT cell biology independent of any antigenic properties.

Oxazolone contains 4 major functional groups: a 5 membered oxazole ring, 2′ phenyl conjugate, 4′-ethoxymethylene and 5′-ketone. In order to isolate the cellular activity of oxazolone, we interrogated MODE-K cells with compounds containing specific deletions/substitutions of key components within the oxazolone structure. In this manner, we were able to identify a natural dietary component 2,4,5-trimethyl-2,5-dihydro-1,3-oxazole (TMO) found in coffee and other food sources [FIG. 1c] [35] which lacks the 2-phenyl ring, 5-ketone, and 4-ethoxymethylene as the minimal structural unit sufficient to confer cellular activity in epithelial cells based on its ability to suppress Mttp and induce CXCL16, similar to that observed with the parent oxazolone structure [FIG. 2]. Importantly, substitution of elements within the oxazole ring itself leads to loss of these biological effects; neither 1-methyl-pyrroline (MP, FIG. 1E) nor 1,2,4-trimethylcycicopentane (TMC, FIG. 1F) could modulate Mttp or Cxcl16 expression. Moreover, MODE-K cells conditioned with oxazolone or TMO, inhibited IL-10 production when co-cultured with an iNKT hybridoma in the presence of lipid antigen α-galactosylceramide (αGC) presented on CD1d surface molecules [FIG. 3A]

We next asked if the isolated oxazole structure was sufficient to induce a colitis phenotype in vivo. Indeed, administration of TMO phenocopies the inflammation associated with oxazolone and leads to increased weight loss, colon shortening, histopathology (based on blind scoring by a pathologist) and expression of Th2 cytokines characteristic of ulcerative colitis[8, 11] compared to vehicle treated control animals[FIG. 5]. We then investigated the broader capacity of OxC compounds to stimulate epithelial transcription changes and influence CD1d restricted iNKT cytokine production. Vinclozolin, a ubiquitous fungicide[28] detectable in human urine [100] demonstrates a similar transcriptional pattern [FIG. 2] and exerts modulatory effects on iNKT cell responses similar oxazolone [FIG. 3]. Likewise, stimulation of lysates from an E.coli strain that produces the OxC B17 peptide also altered CD1d restricted iNKT production of IL-10 compared to the same strain lacking the B17 operon [FIG. 7B].

The data presented herein support a model in which exposure to a broad class of oxazole-ring containing compounds can direct profound transcriptional changes in responsive tissues, such as the epithelium, that concomitantly influence barrier integrity at mucosal sites and modulate iNKT cell inflammatory responses associated with colitis. These data also indicate the presence of cellular sensor(s) that are responsible for recognizing and transducing oxazole dependent signals. Previous studies reveal vinclozolin, one of the lead OxC compounds described herein, as a ligand for the aryl hydrocarbon receptor (AhR),[69]a cytosolic sentinel with nuclear transcriptional capacity that recognizes a diverse set of polycyclic-aromatic hydrocarbons and directs transcriptional activation of the cytochrome P450 xenobiotic-metabolizing enzymes. [67,68] AhR is best known for mediating the toxic effects of dioxins, a class of environmental pollutants found in industrial byproducts and is a toxic contaminant for some herbicides. [70-73] More recently, the AhR has been implicated as a major regulator of intestinal homeostasis and aberrant or deficient AhR activity leads to functional defects in regulatory T cells and Th17 cells as well as depletion of innate lymphoid cells and intraepithelial lymphocytes and host derived AhR ligands promote tolerance to systemic endotoxin challenge. [31,32,34,36,38,77-79] Furthermore, epidemiological studies indicate increased risk for IBD for those living on a diet low in fruit and vegetables, both of which are comprised of high affinity AhR ligands enriched especially in cruciferous foods[5-7] and AhR expression itself has been reported to be differentially expressed in inflamed versus nonaffected tissue of IBD patients.[80] Taken together, these studies provide evidence linking environmental exposures, such as diet, to AhR-mediated mucosal homeostasis.

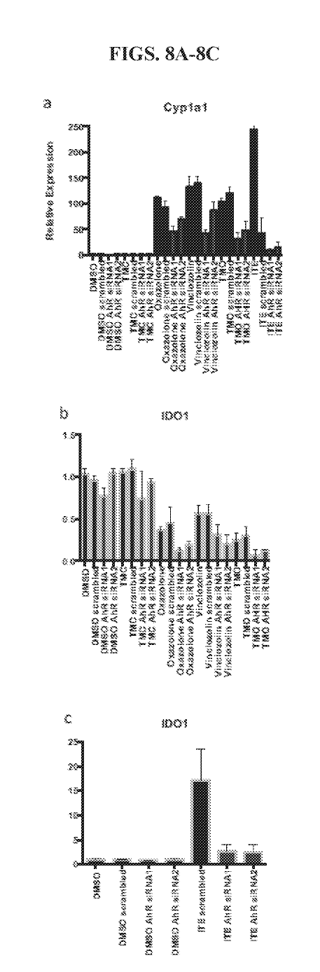

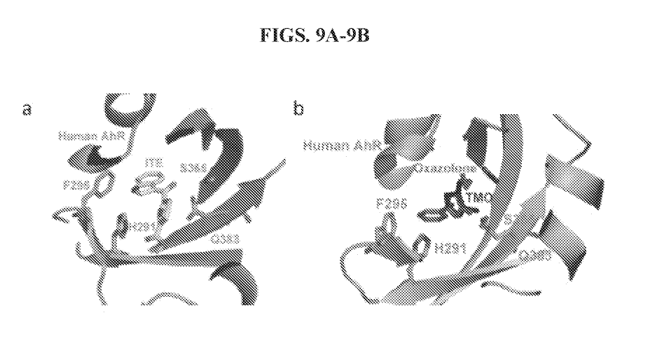

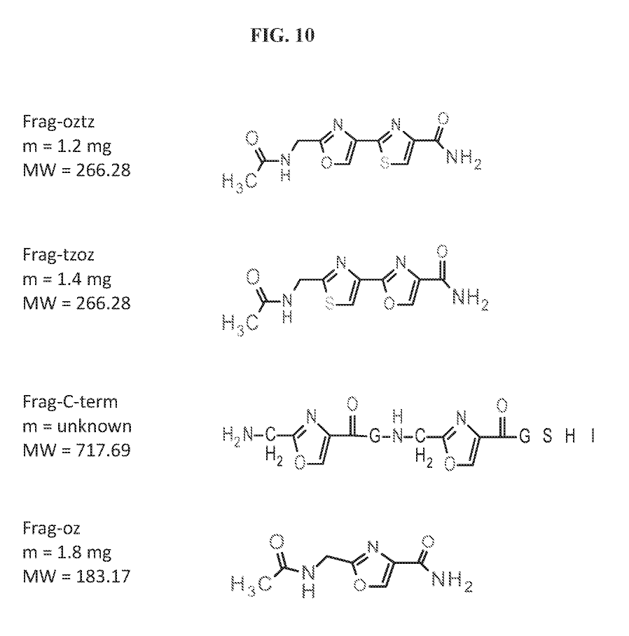

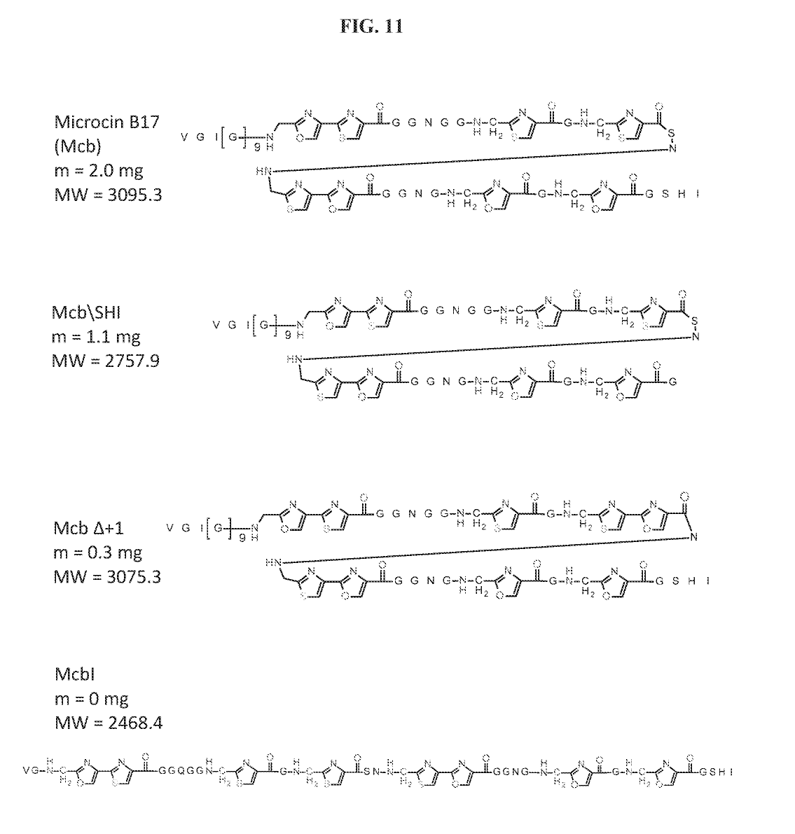

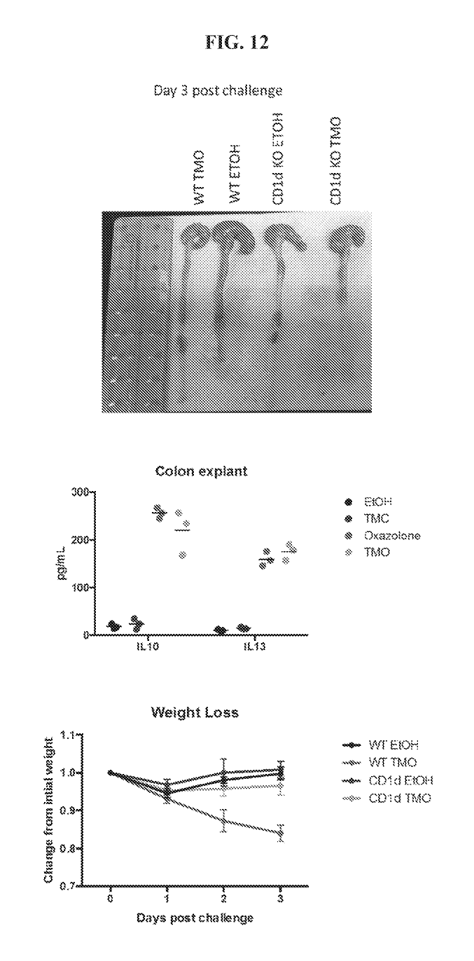

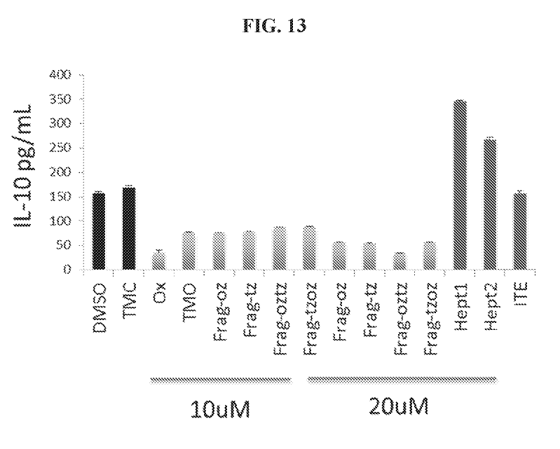

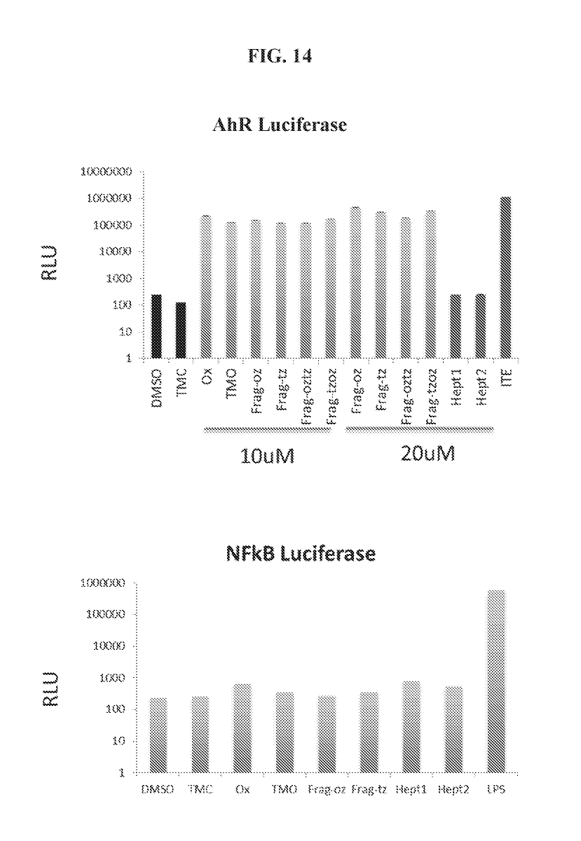

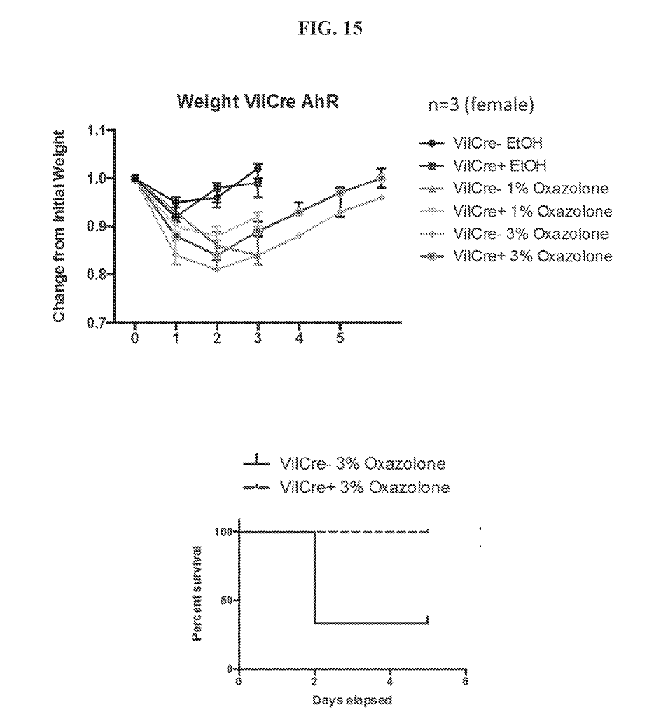

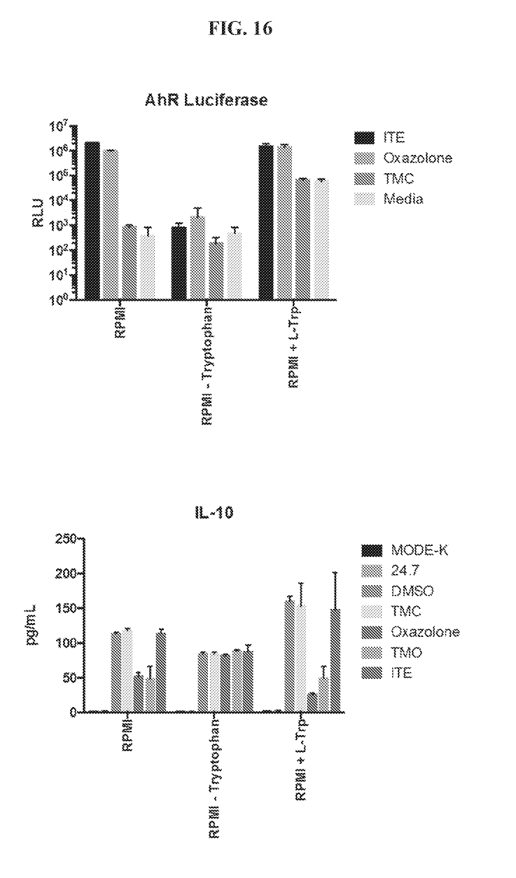

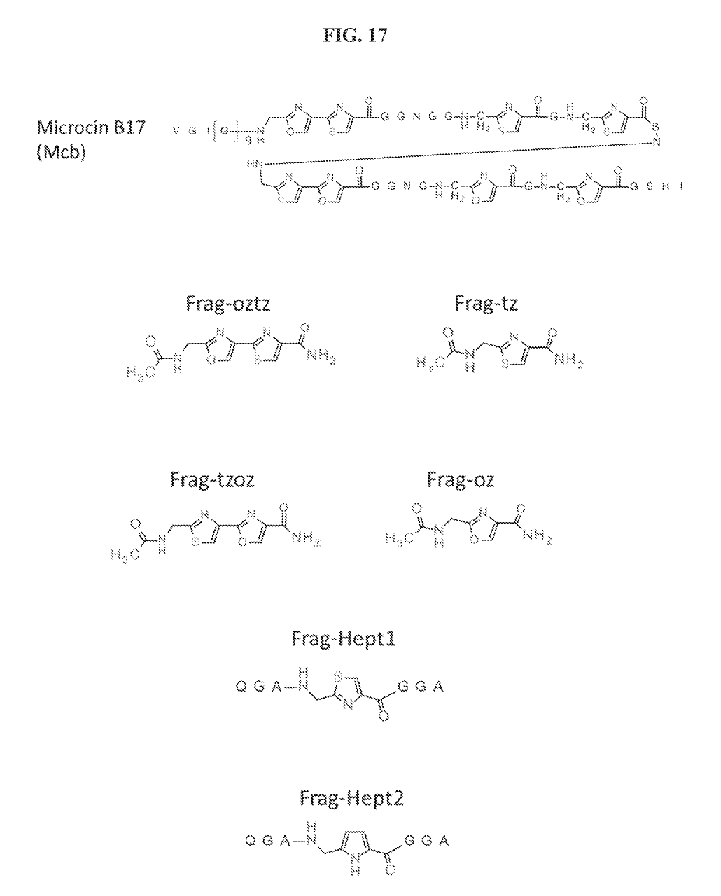

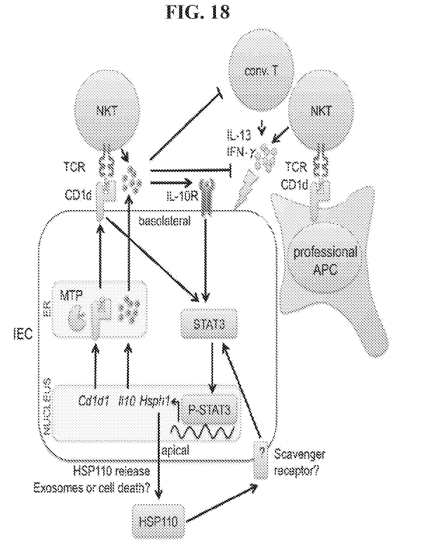

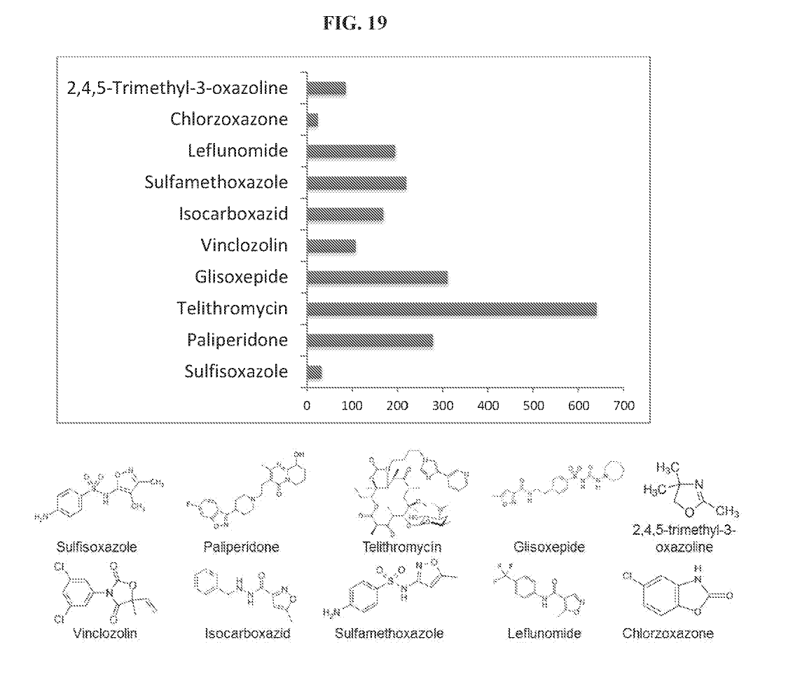

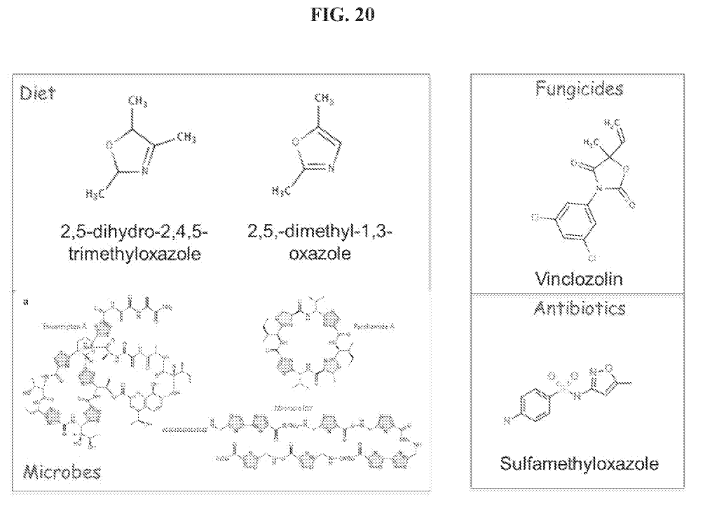

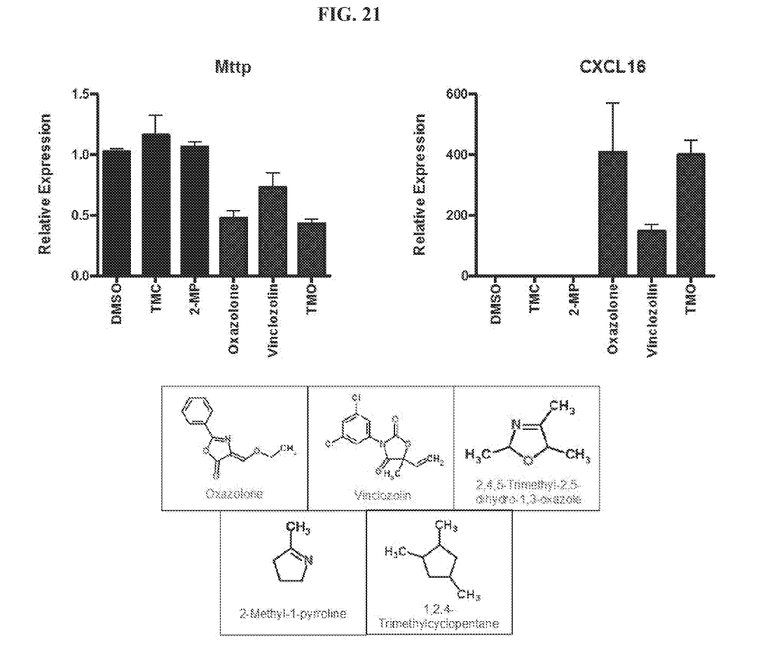

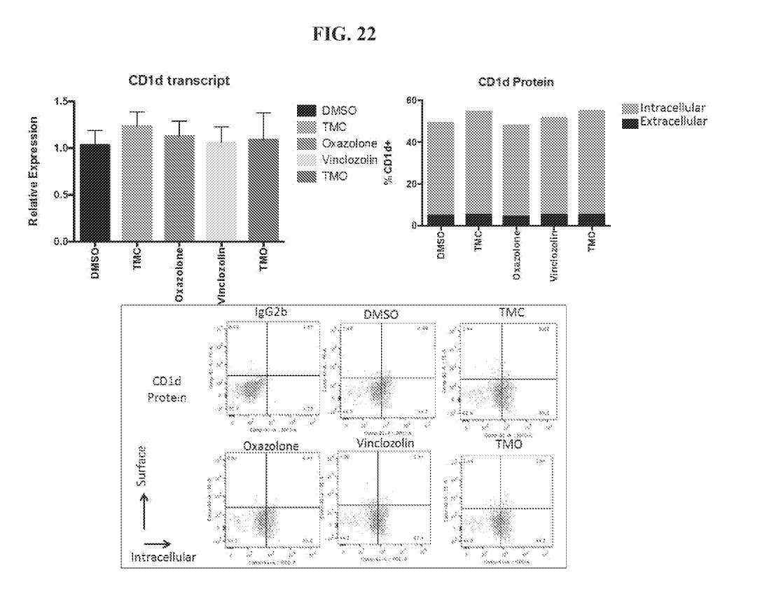

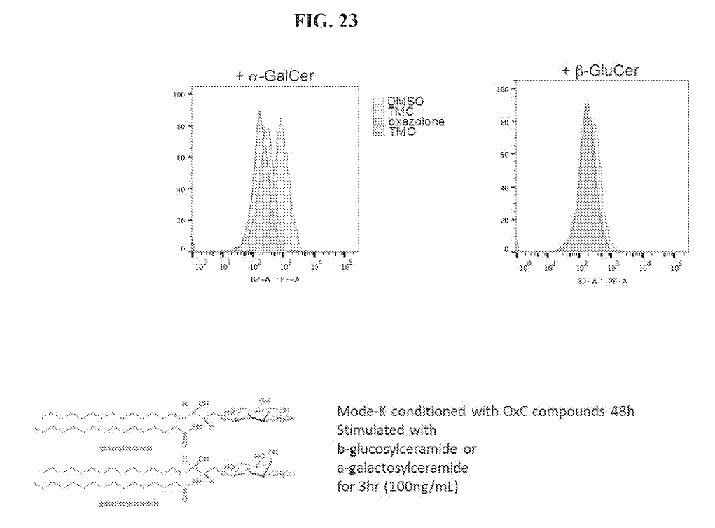

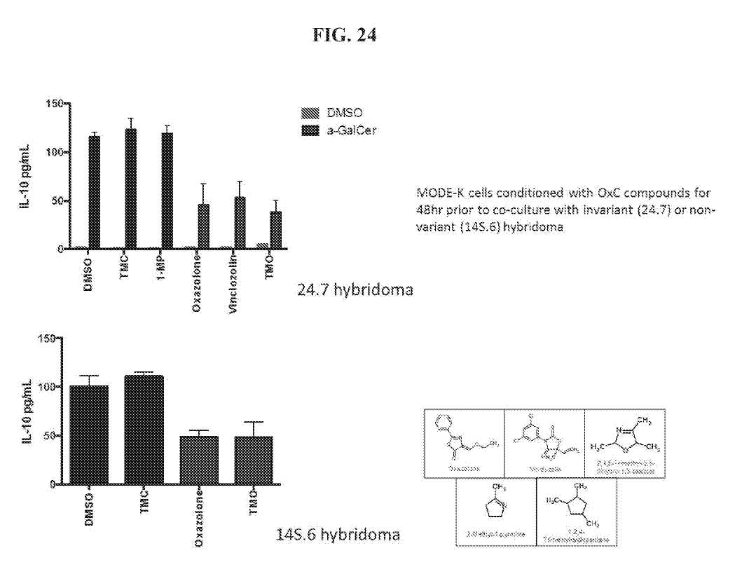

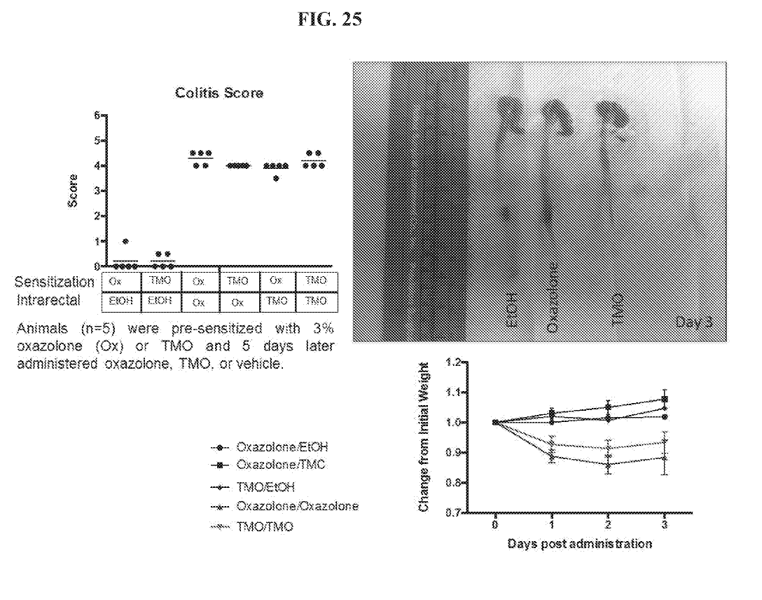

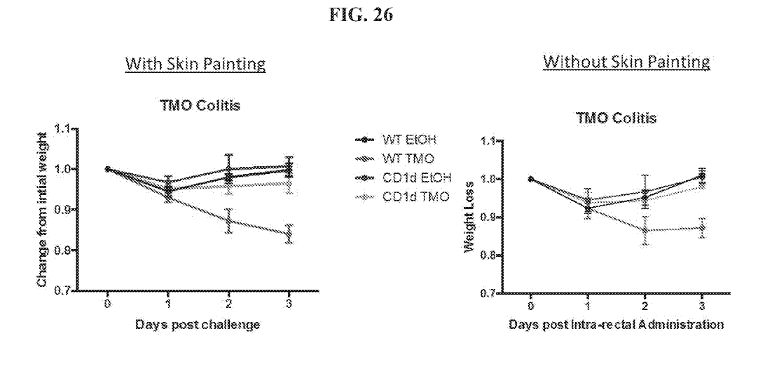

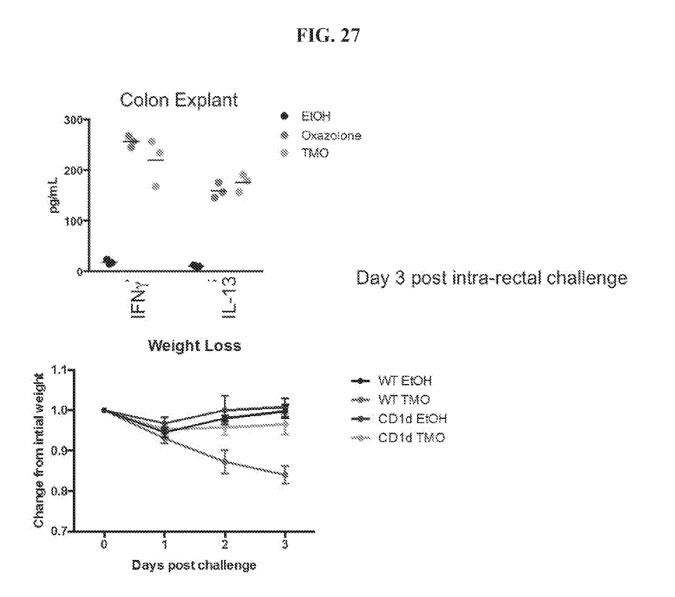

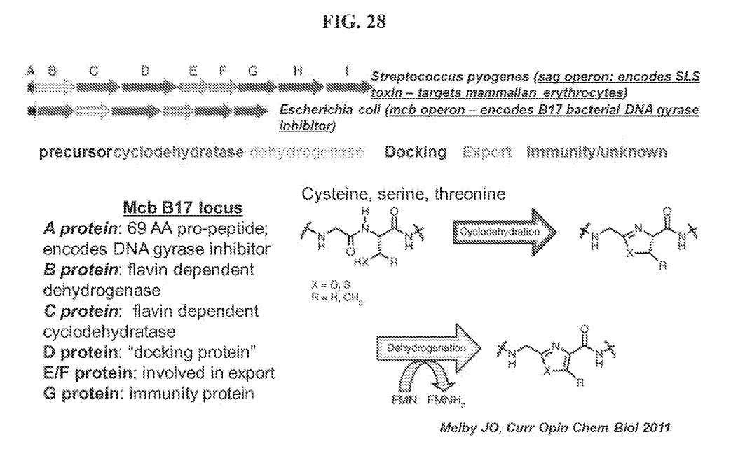

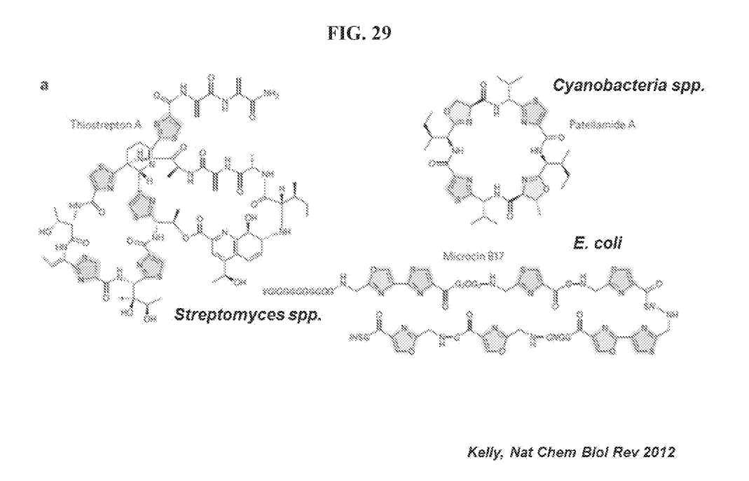

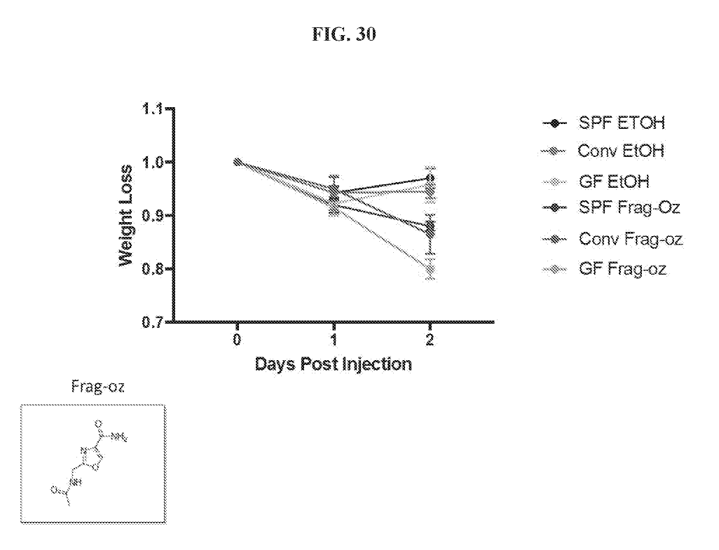

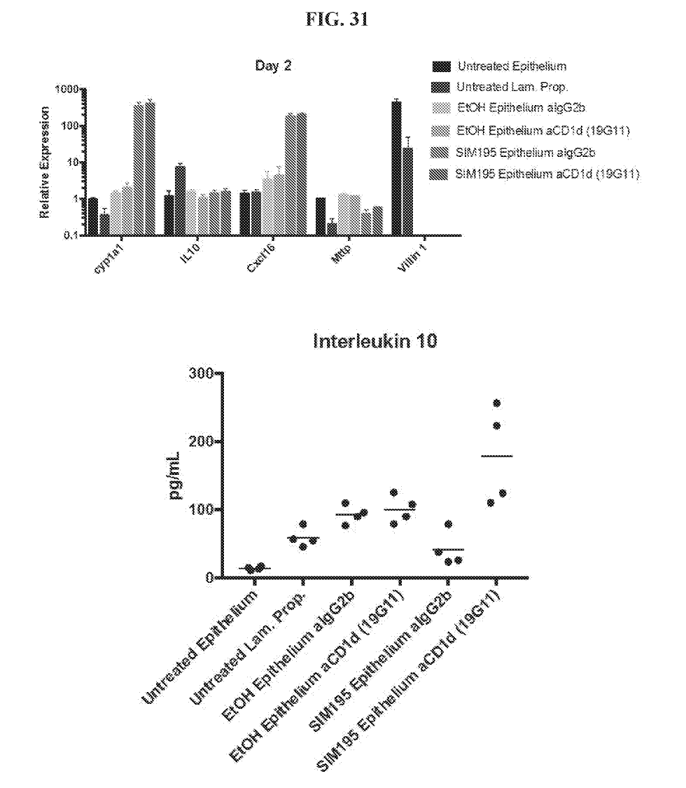

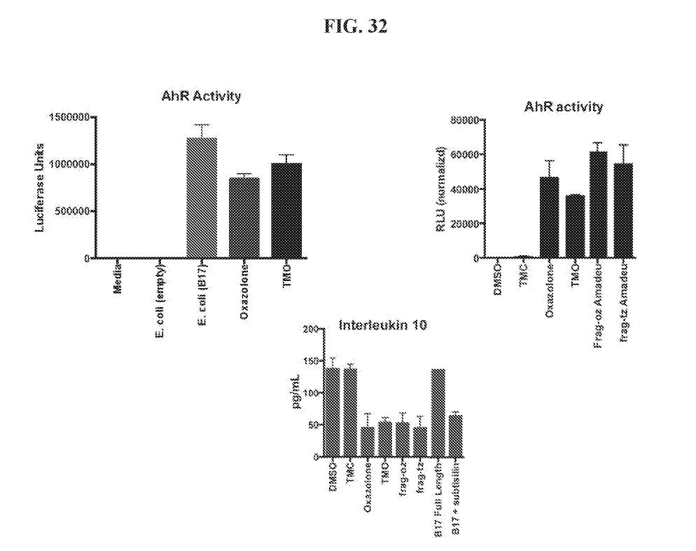

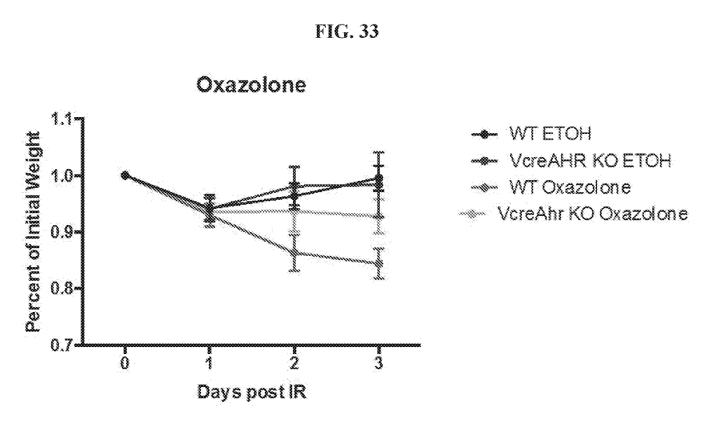

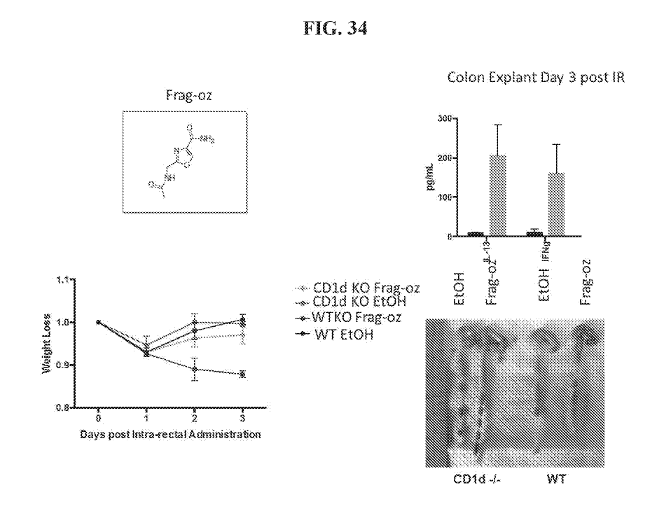

Stimulation of MODE-K cells with OxC compounds but not control compounds differentially regulates two well characterized AhR targets: Cyp1a1,[68] and IDO1. [81] [FIG. 8] Specific siRNA mediated knockdown of the AhR in MODE-K cells diminished expression of both AhR targets in response to OxC stimulation. Moreover, to our surprise, AhR depletion also restored aGCdependent IL-10 production when MODE-K or primary hepatocytes were exposed to OxC—but not control compounds [FIG. 2]. Thus, these data implicate AhR not only as a mediator of epithelial responses to OxC compounds but also indicate that AhR can attenuate iNKT immunomodulatory activity, which is important in intestinal epithelial cell barrier protection, revealing a novel role of AhR in promoting mucosal inflammation in response to OxC exposure.

Defining the structural features underlying colitogenic potential of oxazole-containing compounds derived from microbes, diet and the environment.
Determining minimal structural features of oxazolone required to elicit cellular responses in CD1d bearing epithelial, professional antigen presentation cells and natural killer T cells.

As described herein, we have devised a strategy that systematically evaluates the functional requirements for each moiety within our exemplary OxC compound, oxazolone, in which we stimulate epithelial (MODE-K) and myeloid cell lines (JAWSII, RAW264.7) with oxazolone, TMO (which contains the oxazole ring but lacks the 2′phenyl conjugate, 4′-ethoxymethylene and 5′ketone), MP (contains pyrrole instead of oxazole ring and thus serves as control), TMC (which substitutes carbons at both the 1′-oxygen and 3′-nitrogen positions and thus serves as control) or vehicle control. Transcript and protein production of NKT signatures and CD1d regulated epithelial barrier associated gene targets (Mttp, Cxcl16, CD1d, Hsp110) are assessed and oxazolone conditioning of CD1d bearing epithelial or myeloid APCs on NKT cell responses determined, using both invariant, auto-reactive, or non-variant NKT hybridomas in the presence of absence of model CD1d lipid antigen, α-GC in the case of invariant NKT, by measuring proinflammatory (IL-4, IL-13, IFNγ) and anti-inflammatory (IL-10) cytokine production. Conversely, to assess cellular effects of OxC on NKT activity, primary iNKT cells isolated from colonic LP or peripheral organs are conditioned with OxC or control compounds and co-cultured with MODE-K, JAWSII, or RAW264.7 cells+/−α-GC and cytokine production is measured.

Our data indicate that OxC compounds activate conserved cellular pathways resulting in specific transcriptional profile changes that are biased toward NKT signature genes [FIG. 2] and modulation of NKT response to α-GC lipid presentation by CD1d [FIG. 3]. RNA-seq is used to assess global transcriptional changes in response to OxC compounds compared to control or vehicle in IECs, colonic lamina propria NKT cells, and mucosal APCs. Transcript abundance and differential expression between OxC treated and vehicle control is assessed using DESeq (p<0.01).[83]Thus a comprehensive view of global transcriptome dynamics in distinct mucosal cell types in basal and activated contexts is obtained.

Defining minimal structural features underlying colitogenic potential of oxazole-containing compounds.

The in vitro studies described herein indicate that the colitogenic potential of oxazolone can be resolved to the 5-member oxazole ring structure as observed with TMO. Without wishing to be bound or limited by theory, we submit that exposure to a minimal active structural unit composed of the oxazole ring in mucosal compartments is sufficient to recapitulate tissue-specific cellular pathways leading to disease pathogenesis. Animals are subjected to both acute and chronic regimens of oxazole or TMO and four aspects of colitis development are monitored: weight loss, histopathology, immunological response, and cellular composition of the colonic lamina propria. Colons are subject to blind quantitative histopathology scoring using the following criteria: loss of goblet cells, presence of crypt abscesses, hyperemia in the mucosa, cellular infiltration in the lamina propria elongation of colonic mucosa, and epithelial erosion. The number of conventional T cell, NKT cell and mucosal APC subsets are enumerated by flow cytometry. At empirically determined time points, colonic epithelial and lamina propria mononuclear cells are sorted by FACS and subjected to quantitative real-time PCR analysis for tissue specific OxC targets identified, including Mttp and Cxcl16. In this manner, the structural moieties of oxazolone chemistry responsible for initiating colitogenic signals in physiological context are evaluated and defined.

Establishing a developmental model of colitis based on early exposure to bacterial derived oxazole-containing compounds.

We have identified an oxazole producing commensal bacterial strain that expresses the TOMM B17[15] specifically capable of modulating changes in transcription and CD1d-restricted iNKT responses through epithelial conditioning, similar to what we observe in response to purified oxazolone or OxC compounds alone [FIG. 7]. Colitogenic potential is measured by first purifying the OxC compound B17 microcin from E. coli (recA56 strain). [101] via high performance liquid chromatography (HPLC). Purified fractions are tested first for anti-microbial activity by measuring the capacity to kill a B17 sensitive recA56 that lacks the mcb operon and thus does not express the mcbg gene, which confers resistance against B17. Active fractions are sequenced to verify the identity of the 69 amino acid peptide and liquid chromatography-mass spectrometry performed to quantify the peptide and confirm the presence of oxazole heterocycles at residues Serine 26, Serine 41, Serine 48 and Serine 51 in active fractions.[101, 102] Purified fractions are tested for their ability to induce shared oxazolone/OxC compound targets in epithelial, JAWSII, MODE-K and their ability to modulate CD1d-restricted iNKT response in APC:iNKT hybridoma co-culture assays.The colitogenic potential of B17 in vivo is then assessed by administering this compound or nonheterocycled (lacking oxazole) peptide in our acute and chronic models of colitis, to assess response specifically to the mature oxazole containing form of B17. Gene ontology studies reveal a phylogenetically diverse set of microbes (including commensals) that encode enzymatic machinery consistent with the production of thiazole/oxazole modified microcins (TOMM)[103, 104]. Furthermore, microbial regulated early life events can have profound effects on iNKT accumulation and response in the gut that confer susceptibility to oxazolone colitis during adulthood.[9] Thus, we test whether developmental accumulation and response to microbial derived OxC compounds (i.e. B17) can lead to development of UC-like pathogenesis later in life by monocolonizing animals in germ free isolators with a normally non-pathogenic strain of E. coli (recA56) expressing WT mcb (B17 operon) or strains with mutations in mcbd that have previously been shown to prevent production of mature heterocyclized B17 microcin. Successful colonization of the gut is monitored by PCR of 16S ribosomal DNA and the presence of WT or mutant mcb operon and histological analyses of the colon or colonoscopy performed at 8, 12, 24 and 52 weeks after birth.

Identifying and characterizing molecular sensors of microbial or environmental colitogenic oxazole-containing compounds.

Assessing a role for Aryl Hydrocarbon Receptor in mediating cellular response to OxC compounds. Stimulation of epithelial cells or primary hepatocytes with oxazolone or OxC compounds modulates well-characterized AhR targets, Cyp1a1 and Idol [FIG. 8]. Moreover, depletion of AhR, by siRNA-mediated knockdown or genetic targeting, abrogates activation of these targets and rescues defects in IL-10 production by iNKTs co-cultured with oxazolone or OxC conditioned APCs in the presence of αGC. These findings are validated in bone marrow derived macrophages and DCs and primary epithelial and mucosal APC subsets isolated from WT or AhR-deficient animals and stimulated ex vivo with a panel of OxC compounds, control compounds using the host derived AhR ligand 2-(1H-Indol-3-carbonyl)-4-thiazolecarboxylic acid methyl ester (ITE) as a control.

Expression of AhR or NKT signature genes and additional OxC response genes identified from our global transcriptome profiling are monitored by qPCR. The role of AhR in iNKT cell responses are determined by reciprocal co-culturing of primary WT or AhR-deficient IECs or APCs and WT or AhR-deficient primary colonic lamina propria iNKT cells in the presence or absence of αGC.

Defining the aryl hydrocarbon receptor as a novel sensor for colitogenic oxazole-containing dietary and environmental compounds.

Structural homology modeling of multiple alignments between the sequences of PAS-B human AhR and PAS-B Hif-2 provides an alternative strategy to identify novel candidate AhR ligands given the lack of definitive structural resolution of the AhR ligand binding domain (LBD).[84] The X-ray crystal structure of Hif-2a PASB (PDBID:4GHI) was used to generate the homology model of human AhR-LBD using SWISSMODEL.[85] ITE, oxazolone and TMO were built into this structure and docking orientation within the human AhR-LBD was determined by calculating the best energetically favorable scored solution by RosettaDock [FIG. 9].[86] The oxazole moiety of both oxazolone and TMO are coincident and indicate potential interactions with Histidine 291 (H₂₉₁), Serine 365 (S365), and Glutamine 383(Q383) residues. The phenyl ring of oxazolone can also facilitate potential aromatic (7t) interactions with phenylalanine 295 (F295). Interestingly, while this orientation resembles ligand binding of ITE it differs significantly from the docking orientation of another AhR ligand FICZ.[67,73,88]To validate in silico data, classical AhR binding competition assays are performed using radiolabeled high fidelity AhR ligand TCDD.[84]Hepatic cytosol is prepared from C₅₇B1/6 mouse livers and measurement of AhR ligand binding of oxazolone, TMO and TMC are carried out by autoradiography. In order to carefully characterize the mechanism of OxC ligand recognition by AhR the ability of WT or AhR mutants is assessed with substitution of critical candidate residues within the PAS-B domain predicted by structural homology modeling to activate an XRE reporter plasmid comprised of the Cyp lal enhancer element directly recognized by AhR upstream of the firefly luciferase coding sequence. WT and AhR mutants required for XRE-reporter activity are reconstituted in AhR-deficient bone marrow DCs and further validated by qPCR of defined AhR targets, like Cyp1a1 and IDO1, in the presence of OxC, control compound or vehicle stimulation allowing us to decipher whether sensitivity to OxC compounds is directly mediated by AhR recognition. Binding of AhR to putative transcriptional targets is subsequently validated by chromatin immunoprecipitation.

Evaluating the role of AhR response during oxazolone colitis.

To determine the role of AhR signaling during oxazolone colitis, WT and AhR-deficient animals are administered oxazolone, TMO or vehicle control and colitis development monitored. If AhR is indeed a cognate receptor for OxC, it is expected that AhR-deficient animals are protected against oxazolone and OxC colitis compared to WT animals. AhR-deficient animals are then reconstituted with adenovirus[8] expressing WT AhR or AhR mutants previously determined to be necessary for OxC ligand recognition (i.e. H₂₉₁, S365, Q383) to empirically test which residues within the AhR-PASB domain transduce the colitogenic activity of OxC compounds in vivo. AhR is ubiquitously expressed in both parenchymal and hematopoietic tissue so 2 strategies are employed to resolve tissue specific differences in AhR activity during colitis. First, commercially available AhRfl/fl animals are used to generate animals with specific deletions in epithelial, myeloid, or lymphoid compartments (using tissue specific Cre animals) subjected to oxazolone or OxC colitis. Second, bone marrow chimeras with selective deficiency of AhR in the radio-resistant compartment are generated and OxC responses assessed. In this manner, a role for hematopoietically-derived AhR in a host with sufficient expression of AhR in parenchymal cells in the intestine (and vice versa) is assessed.

Assessing AhR sensing of oxazole containing compounds derived from microbial or environmental sources.

A goal of the studies described herein is to evaluate how environmental signals cue mucosal immunity during the development of IBD. For instance, epidemiological studies have supported important interactions between diet and the risk for developing IBD[6], yet mechanistic links between exposure to specific dietary compounds, the intestinal immune system, and microbiota have been slow to emerge.[89] Signficantly, TMO is a natural dietary compound abudant in coffee and chocolate and OxC compounds have been reported to be major dietary components of at least five food systems.[65] Thus, enriching diet with natural oxazole-ring-containing structures can direct changes in epithelial barrier activity, alterations in microbial communities and modulation of mucosal inflammation associated with development of IBD (in genetically susceptible host).

Without wishing to be bound or limited by theory, we hypothesize that the colitogenic activity of OxC dietary supplementation is mediated by AhR, a cell intrinsic receptor that has been previously reported to sense phytochmicals and tryptophan derivatives enriched in cruciferous vegetables.[68] WT (IL10fl/fl) or recently described ILlODIEC mice[8,82]with are fed either standard diet (5058 Autoclavable Rodent Lab Diet) or a synthetic diet (AIN-76A Purified Rodent Diet) depleted of AhR ligands. [32] Cohorts of animals are supplemented with TMO or the noncolitogenic compound TMC. Intestinal barrier permeability is monitored by measuring FITC-conjugated dextran (FD4) in blood after administration by gavage. Changes in microbial communities are evaluated by qPCR analysis of 16S ribosomal DNA (Firmicutes, Actinomyces, Proteobacteria, Bacteroides) to detect global changes in microbiota-associated phyla in feces. Development of colitis is assessed by monitoring weight changes and mouse colonoscopy. Animals are sacrificed and evaluated for histopathology, immunological response, and cellular composition of the colonic lamina propria, and evidence of bacterial translocation defined by colony forming unit assays in mucosal lymphoid tissue (i.e. mesenteric lymph node). In addition, a role for AhR is evaluated by administering normal or synthetic feed supplemented with TMO or TMC to WT and AhR-deficient animals. Properties of intestinal inflammation and dysbiosis are monitored and specific AhR responses measured, including Cyp1a1 expression and tryptophan depletion by measuring the ratio of tryptophan:kyneurenine in the serum by clinical chemistry.[73] As a control, indole-3-carbinol (I3C) is administered a known AhR dietary ligand with no previously defined colitogenci potential to evaluate AhR response to dietary supplementation. If successful, similar studies are performed with mice raised under SPF and GF conditions to define the role of the microbiota.

Oxazolone, TMO, and non-oxazole containing compounds controls (MP, TMC) are dissolved in dimethyl sulfoxide and added to MODE-K, JAWSII, and RAW264.7 cells. Kinetic transcript profiles of Mttp, Cxcl16, CD1d, and Hsp110 are assessed by qPCR normalizing transcript abundance to house keeping gene, (3-actin. Protein expression is quantified by immunoblot (MTTP, HSP110) or ELISA (CXCL16). CD1d protein expression is monitored using monoclonal antibodies conjugated to phycoerythrin and fluorescein isothyocynate to assess CD1d trafficking to and from the cell surface.[89]Stimulatory ligands are washed out prior to administration of αGC, and conditioned cells are co-cultured over night with 4 classes of invariant (DN32.D3, 24.7, 24.8) or non-variant (14S.6) to assess αGC specific and autoreactive responses to epithelial and myeloid cell lipid presentation. Production of IL-10, IL-4, IL-13, IFNγ response to epithelial and myeloid, and the source of cytokine production (APC or NKT) are determined using gluteraldehyde fixation. [89] Primary iNKT cells are isolated from colonic lamina propria or peripheral organs (liver, spleen, mesenteric lymph node) and purified by FACS (BD ARIA-II Sorter) using monoclonal antibodies against CD45, TCRI3, CD3E, and CD1d:αGC loaded tetramer (APC conjugated) and exclusion of unloaded CD1d tetramer (PE). Primary iNKTs are conditioned with oxazolone, a panel of OxC compounds or our controls and cocultured with naïve MODE-K, JAWSII, and RAW264.7 cells +/−αGC to assess specific and autoreactive responses by monitoring interleukin production. For transcriptome studies, RNA from oxazolone, OxC compound or control compound stimulated MODE-K, JAWSII, and RAW264.7 cells are isolated, depleted of rRNA and subject to polyadenylated RNA enrichment by negative selection. Strand specific libraries are generated using 500 ng RNA input and subject to paired-end 100 bp read sequencing using a HISEQ2000 at the Dana Farber Cancer Institute. Sequence reads are aligned to the mouse mm10 reference genome using Tophat alignments restricted to uniquely mapping reads with <2 possible mismatches permitted. Differential transcript abundance between vehicle, control, OxC compound treated, or oxazolone are quantified as RPKM values using a DESeq threshold p<0.01. Candidate transcripts arevalidated in primary IECs (EpCAM+ CD45−) or mucosal APCs by qPCR.

8 week C₅₇B1/6 animals housed under SPF conditions are subjected to acute or chronic administration of oxazolone, TMO, TMC, or EtOH vehicle. In acute oxazolone, animals are given a high dose (5% by volume) of the experimental compound.[10] During chronic oxazolone colitis, animals are first sensitized by direct application of a 3% oxazolone or OxC compound solution to the skin followed by intra-rectal administration of a 1% oxazolone or OxC compound solution dissolved in 50% EtOH.[12] Changes in weight are monitored and colons harvested on day 3 and day 5 post-rectal challenge for histopathological analysis. Distalhyperemia in the mucosa, cellular infiltration in the lamina propria, elongation of colonic mucosa, epithelial erosion. The number of conventional T cells, NKT cells and mucosal APC subsets are enumerated by established flow cytometry protocols.[11] In addition, kinetic cytokine profiles are analyzed to verify that OxC colitis phenocopies molecular signatures associated with oxazolone colitis. 1 cm radial tissues are sectioned from distal, medial, and proximal (relative to cecum) colon are incubated overnight in RPMI supplemented with antibiotics and assessed for IL-4, IL-13, IFN and IL-10, all of which are robustly induced during oxazolone colitis. [8] In addition, colonic IECs and lamina propria mononuclear cells are sorted by FACS and analyzed for Mttp and Cxcl16 expression by qPCR. Finally, animals are utilized with targeted disruption of CD1d and Ja18, each of which are resistant to oxazolone colitis due to a depletion of iNKT response to verify that the colitogenic capacity of OxC compounds is fundamentally driven by mucosal NKT responses.

To purify B17 microcin, B17 expressing or non-expressing strain of E. coli (recA56) [101] are inoculated in M63 glucose media (Sigma) and harvested during logarithmic phase. Cell pellets are lysed by boiling in 100 mM acetic acid/lmM EDTA and lysates are vacuum dried at 90° C. and resuspended in trifluoroacetic acid (TFA). This fraction is loaded onto a C₁₈ column on an HPLC apparatus and eluted with an 8-23% gradient of acetonitrile in 0.1% TFA. Purified fractions are collected (Absorbance 245 nm) and tested for anti-microbial activity via critical dilution method.[101]recA56 strain with sensitivity to B17 (due to lack of expression of the mccg immunity gene[101]) are freshly seeded on M63 glucose minimal agar plate and serial dilutions of each fraction are spotted and plates incubated overnight and quantified by colony forming units. Those fractions able to to inhibit >1000 fold compared to lysate from B17 negative recA56 are subject to amino acid sequencing and HPLC-MS (Agilent C₁₈ column connected with Thermo Scientific LTQ-XL) to verify the sequence identity and presence of oxazole heterocycles at Serines 26, 41, 48, 51. Active B17 fractions are then tested against oxazolone, OxC compounds (TMO, vinclozolin) and control compounds (TMC, MP) for epithelial transcriptional changes (Mttp, Cxcl16) and iNKT hybridoma response when co-cultured with OxC conditioned MODE-K, JAWSII, RAW264.7 +/−αGC. The ability of purified B17 to induce colitis is tested in acute and chronic murine models. Heterocyclized or non-heterocyclized (purifed from recA56 expressing mutant mcbd or point mutations at S26,S41, S48 and S51) are tested side by side to verify that colitogenic activity is due to the presence of oxazole rings in the peptide. For monocolonization, 8 week germ free animals housed at the gnotobiotic core are subject to inoculation with recA56 expressing WT, or mcbd mutant MCB operon by gavage and co-housed with outbred “colonizers” in individualzed ventilated cages in separate microisolation chambers as described.[8, 39] Monocolonization is confirmed by quantitative PCR of 16S ribosomal DNA on feces and cecal content. After establishing monocolonized populations, animals are monitored for spontaneous colitis at specific time points during development to assess the effects of developmental exposure to B17 microcins. In addition expression of NKT signature genes (i.e. Mttp, Cxcl16) and CD1d restricted responses from primary iNKTs taken from colonic lamina propria or peripheral tissues is assessed as well.

IECs, BMDMs, and BMDCs, from WT and AhR-deficient animals are isolated and stimulated with a panel of OxC compounds, control compounds or vehicle only. These cells are also stimulated with ITE, a high affinity AhR ligand that activates cis AhR response elements (XRE) at picomolar concentrations to demonstrate the fidelity of AhR-deficient cells. [68] Kinetic transcript analyses by qPCR are performed on known AhR targets (Cypa1, Ido1) as well as novel oxazolone/OxC shared targets identified from RNA-seq. Inflammatory cytokine production is characterized by reciprocal co-culturing of primary oxazolone/OxC compound or control compound conditioned WT or AhR-deficient IECs/APCs isolated with NKT hybridomas +/−αGC or conditioned WT or AhR-deficient primary colonic LP iNKTs co-cultured with MODE-K, JAWSII, or RAW264.7 using flow cytometry based cytometric bead assays.

For AhR binding assays, hepatic cytosol is prepared from mouse livers in HEDG buffer as previously described. [105] Cytosol (5 mg protein/mL) is incubated with 5 nM [3H] TCDD (37 Ci mmole-1) in the presence of DMSO, TCDF, TMO, or TMC for 1 hr at 4 oC. Unbound radioligand is removed with dextran-coated charcoal and samples are subjected to centrifugation against a 10-30% sucrose (v/v) gradient and the presence of radioactivity in each fraction is determined by liquid scintillation. The specific binding is calculated as a function of the equilibrium inhibitor constant Ki=IC50/(1+[L]/Kdl where L designates the radioligand. For transfection studies, reporter activity is measured as the ratio of firefly luciferase to renilla luciferase to control for internal transfection efficiency.

8 week C₅₇B1/6 or Ahr-/- mice (Taconic USA) are housed under SPF conditions in a barrier facility. Ahr-deficiency is associated with defects in cryptopatch and isolated lymphoid follicle development in the gut, which is associated with defects in intestinal lymphoid cell (ILC) development.[34-36] Thus, it is imperative to monitor any developmental defects in mucosal APC or NKT subsets in the colonic lamina propria or in peripheral organs (liver, spleen, thymus, mesenteric lymph node) using flow cytometry strategies described earlier. CD1d, MTTP, HSP110 and IL-10 expression are quantified on IECs and mucosal APCs in WT or AhR-deficient animals. Acute and chronic administration of oxazolone, TMO, TMC or vehicle control is performed as described herein. In order to investigate the role of critical residues within the ligand binding domain of AhR during oxazolone colitis, human AhR ORF from HepG2 cells serially cloned into pAdHM15RGD-CMV6-IRES2-eGFP is amplified, which allows for detection of eGFP in infected tissue (E. coli (3-galactosidase/AdLacZ is used as a control virus). Plasmids are transfected in 293 cells and adenovirus purified by cesium chloride density centrifugation followed by dialysis against Tris-Cl. Plaque forming units and GFP are monitored to determine the infectious dose. Subsequently, AhR adenoviruses are generated with specific mutations in key residues identified from homology modeling and site directed mutagenesis studies described herein. Expression of these constructs is verified in MODE-K and IEC isolated from WT and AhR-deficient animals infected with adenovirus at an MOI of 5. The expression of Cyp1a1 in reconstituted cells is then assessed to verify fidelity of AhR response to OxC compounds or ITE. Finally, WT or AhR animals are infected with Ad:AhR, Ad-Ahrmut or AdLacZ and administered oxazolone. IECs, APCs, and NKTs are isolated from the colon and qPCR performed on NKT signature genes and AhR target genes. For bone marrow chimera experiments, C₅₇B1/6 or Ahr-deficient recipients receive total body irradiation of 1100 rad in two separate doses 4 h apart. The next day 0.5×10⁶ bone marrow cells are delivered from WT or AhR-deficient donors intravenously. After reconstitution, animals are challenged with Oxazolone, TMO, TMC or vehicle control. Body weight is measured daily and evidence of colitis is assessed in blinded fashion using criteria described herein.

After weaning, animals are given normal (5058 Autoclavable Rodent Lab Diet) or synthetic purified diet (AIN-76A purified rodent diet) for 3 weeks. Cohorts are supplemented with 200 ppm of TMO or TMC (Sigma). Changes in intestinal epithelial permeability are measured by the appearance in blood of FD4 administered at 400 mg/kg body weight by gavage in sterile PBS. Blood samples are obtained at 2 and 5 hr after administration and the concentration of fluorescein is determined by spectrophotofluorometry with an excitation wavelength of 485 nm and an emission wavelength of 530 nm. Specific properties of mucosal dysbiosis are analyzed in response to specific dietary supplementation. Bacterial translocation to sterile tissue is detected by CFU assays in the colon-draining mesenteric lymph node. To determine changes in intestinal bacterial load and composition ribosomal 16S DNA of major phylogenetic microbial communities (Firmicutes, Actinomyces, Proteobacteria and Bacteroides) are analyzed by qPCR. To evaluate modulation of intestinal homeostasis and IBD phenotypes by specific diet supplementation via the microbiota, animals are administered the broad-spectrum antibiotic Enrofloxacin (Baytril, Bayer) prior to dietary changes. Any observations dependent on microbial presence are validated in germ free mice maintained in vinyl isolators within the animal facility.

The oxazolone colitis model has been an invaluable tool for studying the genesis of colitis and the development of potential therapeutic strategies against IBD, and importantly, manifests important histological features of ulcerative colitis (such as specific inflammation of only the distal colon)[11] that distinguish it from other models, such as TNBS colitis. In addition, the pathophysiology of oxazolone colitis, such as dependence on NKT derived inflammation and induction of TGFO, are consistent with clinical observations of specimens from patients with ulcerative colitis compared to healthy controls or Crohn's disease patients.[8,11,106] Oxazolone colitis was first developed as an alternative to TNBS colitis[8] and was presumed to be a classic delayed-type hypersensitivity response mediated by T cells responding to “hapten-modified self antigen.” However, to date, unlike in TNBS, the formation of oxazolone conjugated self antigens remains to be detected, and no evidence oral tolerance against oxazolone colitis has been reported.[107]

Preliminary studies have resolved cellular response to oxazolone-like structures containing the oxazole ring. Our systematic approach carefully interrogates the major functional moieties comprising the prototype oxazolone structure enabling us to resolve minimal and sufficient structures conferring cellular response. Moreover, the panel of compounds tested includes natural compounds found in diet (i.e. TMO) and industry (vinclozolin). A major goal of this work has been to develop a molecular framework that defines how environmental exposures (via, inhalation, ingestion or absorption) microbial derived products triggers IBD development in genetically susceptible hosts that derives from a novel class of chemical agents.

To this end, we have queried the Tox21 database of environmental metabolites developed by the Environmental Protection Agency for biological activity attributed to conserved structural moieties within oxazole-containing compounds. Using TMO as our template, the Human Metabolome database (HMDB) was searched for compounds exhibiting a Tanimoto Similarity Score >0.7 yielding 136 molecules. This list was further refined by individually comparing each molecule to TMO and structure comparison having a Euclidean Dissimilarity Score of >0.9 were defined as potential candidates that modulate transcriptional changes and NKT responses through conserved oxazolone response pathways. Thus, we identified two compounds, the anti-fungicide Vinclozolin, anti-mycotic agent fluorinated oxazolone (Fl-Ox) both of which appear to elicit similar cellular responses as oxazolone and TMO in preliminary experiments [FIGS. 2, 3] and the Escherichia coli derived heterocyclic oxazole/thiazole peptide B17 [FIG. 7]. In addition, oxazole related compounds were selected from Super Natural II, a database of natural products, and Pubchem. Natural compounds were selected based on similarity/dissimilarity scores (e.g., Tanimoto, Nams) and dissimilarity (distance) scores (e.g., Euclidean score). Thus, it is important to test a cohort of oxazolecontaining compounds derived from a variety of biological and industrial sources to extrapolate the in vitro structural definitions to colitogenic capacity in physiological context described herein.

Transcriptional profiling of 3 major cellular mediators (epithelial, myeloid APC subsets, iNKTs) of mucosal inflammation provide a highly informative data set that potentially explains distinct tissue-specific physiological response to oxazolone or OxC compounds. In vivo administration of oxazolone is associated with defects in epithelial barrier activity and mucosal dysbiosis leading to CD1d-mediated NKT inflammatory response. Thus, oxazolone or OxC treatment of epithelial cells can incur a transcriptional profile consistent with attenuating CD1d-retrograde signaling and promote CD1d-dependent lipid antigen presentation capacity on professional APCs. There can also be shared transcriptional targets like CXCL16, which has been linked to both hematopoietic and parenchymal cellular sources for mucosal NKT recruitment. [8] The data described herein indicate that oxazolone can modulate NKT responses through indirect cellular responses on CD1d bearing cells. Whether oxazolone and OxC compounds directly modulate NKT responses is of significant interest.

A potential confounding factor in oxazolone colitis (and TNBS colitis) is the use of 50% ethanol as a solvent, which when administered alone can introduce tissue damage. This has motivated us to develop more physiologically relevant experimental models, such as a microbial or dietary delivery system of oxazolone containing compounds. Our model of monocolonization allows us to evaluate if commensal derived OxC compounds can elicit a colitic phenotype. Moreover, given our recent findings emphasizing the importantance of early life events on iNKT accumulation in the gut, [8,39] this model can prove to be an invaluable tool for studying developmental aspects of exposure and accumulation of compounds with colitogenic potential in a genetically susceptible host. It may be found that simply the presence of the B17 microcin is not sufficient to induce colitis in WT animals, most likely due to the integrity of mucosal barrier function. This possibility is investigated by colonizing animals with specific deletions of IL-10/CD-1d in the epithelial compartment, which has previously shown to compromise epithelial barrier function[8].Monocolonization studies are approximated using sulfatrim[8] followed by oral gavage of B17 expressing, mutant or non-expressing E. coli. If successful, re-derivation of these animals in our germ free facility and monocolonization studies are performed.

The microbiota plays a vital role in the development of IBD and seminal studies have revealed specific commensal bacteria that can drive or attenuate inflammation, such as Provetella spp. or Bacteroides fragilis, [108, 109] respectively, however the need for additional microbial models is evident (Frank et al. 2007, Naumgart et al. 2007, Issa et al. 2007, Prindiville et al. 2000). In addition, Olszak et al. Science 2012 showed a response to oxazole compounds regulated by early life microbial colonization. The model described herein is advantageous in that the molecular mechanism responsible for generation of B17 microcins has been well established allowing us to easily purify the compound for in vitro and in vivo studies, as well as take advantage of mutant strains that validate the specificity of the B17 microcin as the causative agent of colitis. Given some evidence linking certain strains of E. coli to IBD development, this model is useful not only to study the physiological effects of OxC compounds on development of IBD, but also as a prototype for studying the role of “pathobionts” in the gastrointestinal tract.

Data indicates a novel role for epithelial-sourced AhR as a modulator of responses to OxC compounds that leads to significant modulation of CD1 mediated NKT responses. AhR can act as a cognate sensor for Ox-containing compounds based on defects in the expression of AhR targets Cyp1a1 and IDOL in AhR depleted MODE-K cells after OxC stimulation. Thus, defects in IL-10 production by NKTs cocultured with OxC conditioned epithelial cells may be a consequence of AhR dependent suppression of an MTP:CD1d:IL-10 pathway previously defined as a major regulator of ntestinal inflammation by the epithelium. [8] Expression of E. coli B17 microcin associated with modulation of NKT response to CD1d lipid antigen presentation was shown. Bacterial (recA56) lysates were incubated with MODE-K cells and cocultured with 24.7 hybridoma in the presence of a-galactosyl ceramide.

The mechanisms underlying such an interaction and the implications of such interactions on mucosal immunity are further investigated herein.

It is also possible that downstream metabolites generated in response to Oxazolone or OxC dependent AhR activation modulate NKT responses supporting a role for AhR as a major regulator but not a de facto sensor of OxC activity. This latter model was demonstrated in an analogous context of APC modulation of regulatory T cell (Treg) development and Th17 function[30-35]. Here, IDO₁ expressing APCs metabolize tryptophan to kyneurenine (KYN) metabolites to drive de novo differentiation of Foxp3+ Tregs through AhR recognition of KYN on naïve CD4+ T cells. Conversely KYN sensing by AhR expressing Th17 cells attenuates IL-17. Moreover, cytochrome P450 activity is associated with metabolism of catabolic AhR ligands, such as dioxins, so prolonged Cyp1a1 can attenuate AhR responses a means of feedback regulation.[67] In these last two instances, AhR-deficiency may not restore MTP expression in epithelial cells. Thus, transcriptional profiling can be used to identify novel candidate sensors.

Recent emerging work has implicated AhR in regulating intestinal homeostasis through regulation of at least 4 cellular targets: regulatory T cells, Th17 cells, intestinal epithelial lymphocytes,[34] intestinal lymphoid cells [30-32]. AhR-deficient mice undergo normal lymph node formation during embryonic development but exhibit defects in the formation of cryptopatches and isolated lymphoid follicles in the gut during adulthood [33-36]. Specific defects in the development of IECs and IL-22 producing ILCs have also been reported implicating AhR in the regulation of epithelial homeostasis and mucosal immunity [31]. The specific role of IECs and ILCs during oxazolone colitis has not been specifically investigated. Thus, IL-22, IgA and the expression of RegIIIγ (ILC markers) and the proportion of γδ and CD8αα positive T cells in the colonic lamina propria in WT and AhR-deficient animals subjected to oxazolone colitis are monitored to determine whether these factors play a role in mediating intestinal inflammation. In these studies, OxC compounds can direct their colitogenic activity through AhR sensing and dietary exposure is proposed as one possible physiologically relevant route of effect. Utilizing normal or synthetic defined diets supplemented with specific candidate ligands provides an experimental model to determine whether the specific activity of TMO is sufficient to induce colitis when provided in diet. Supplementation of diet with TMC that lacks oxazole ring structure is an important control for ligand specificity. Determining the specificity of ligand responses by AhR is also critical for development of this experimental model and AhR-deficient can be insensitive to the colitogenic activity of TMO supplemented minimal diets. The use of I3C which has been previously utilized in other studies as an AhR activating supplement but confers tolerogenic signals to the intestine and reveals important insight into the heterologous nature of AhR recognition and further evidence of how dietary composition can modulate mucosal immunity.

The various methods and techniques described herein provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, a person of ordinary skill in the art will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A pharmaceutical composition comprising an inhibitor of an Oxazole containing (OxC) compound and a pharmaceutically acceptable carrier, wherein the OxC compound is a compound of any of Formula I 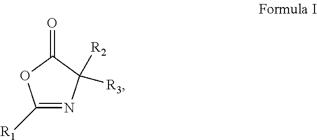
Formula II 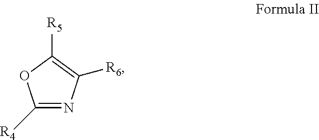
Formula III 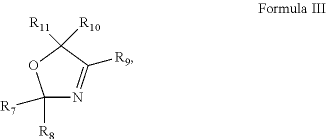
or Formula IV 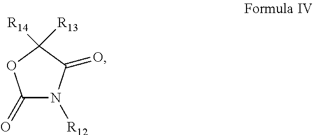
wherein R₁-R₁₄ are each independently selected from the group consisting of hydrogen, alkyl, alkenyl, alkynyl, aryl, heteroaryl, alkoxy, amino, and carbonyl, provided that each of Formulas I-IV has two or more R groups which are not hydrogen.

2. The pharmaceutical composition of claim 1, wherein the compound of Formula I is Oxazolone

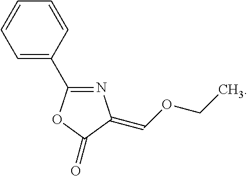 [SMILES: CCO/C=C1\N=C(c2ccccc2)OC1=O]

3. The pharmaceutical composition of claim 1, wherein the compound of Formula II is selected from

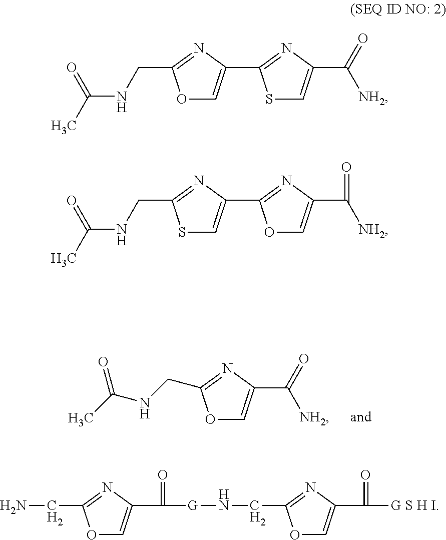

4. The pharmaceutical composition of claim 1, wherein the compound of Formula III is 2,4,5-trimethyl-2,5-dihydro-1,3-oxazole (TMO):

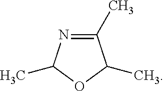

5. The pharmaceutical composition of claim 1, wherein the compound of Formula IV is vinclozolin:

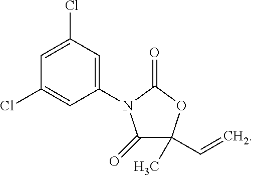 [SMILES: C=CC1(C)OC(=O)N(c2cc(Cl)cc(Cl)c2)C1=O]

6. The pharmaceutical composition of claim 1, wherein the OxC compound of Formula II is a thiazole/oxazole-modified microcin (TOMM).

7. The pharmaceutical composition of claim 6, wherein the TOMM is microcin B17 or mutants or fragments thereof.

8. The pharmaceutical composition of claim 1, wherein the inhibitor of an OxC compound specifically binds to the OxC compound, its metabolites, or a metabolic product induced by the OxC compound.

9. The pharmaceutical composition of claim 8, wherein the inhibitor of an OxC compound specifically binds to the OxC compound and inhibits or prevents binding of the OxC compound, its metabolites, or a metabolic product induced by an OxC compound to the Aryl Hydrocarbon Receptor (AhR) of SEQ ID NO: 1 and its activation.

10. The pharmaceutical composition of claim 9, wherein the inhibitor of an OxC compound inhibits or prevents binding of the OxC compound, its metabolites, or a metabolic product induced by an OxC compound to one or more amino acids selected from H₂₉₁, F295, S365, and Q383, thereby inhibiting AhR binding to an OxC compound.

11. The pharmaceutical composition of claim 1, wherein the inhibitor of an OxC compound is an Aryl Hydrocarbon Receptor (AhR) antagonist.

12. The pharmaceutical composition of claim 11, wherein the AhR antagonist binds to the Aryl Hydrocarbon Receptor (AhR) of SEQ ID NO: 1 at one or more amino acids selected from H₂₉₁, F295, S365, and Q383 of SEQ ID NO: 1, and inhibits or prevents AhR binding to an OxC compound, its metabolites, or a metabolic product induced by an OxC compound.

13. The pharmaceutical composition of claim 1, wherein the inhibitor of an OxC compound is an antibody or antigen-binding fragment thereof

14. The pharmaceutical composition of claim 13, wherein the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)₂ fragments, a single chain antibody molecule, a diabody or a linear antibody.the antigen-binding fragment thereof that that specifically binds to the OxC compound is a Fab fragment, a Fab′ fragment, an Fd fragment, an Fd′ fragment, an Fv fragment, a dAb fragment, isolated CDR regions; F(ab′)₂ fragments, a single chain antibody molecule, a diabody or a linear antibody.

15. The pharmaceutical composition of claim 1, wherein the inhibitor of an OxC compound is a small molecule.

16. A method of treatment of a disease or disorder associated with epithelial barrier integrity and/or iNKT cell-mediated inflammatory responses, comprising administering to a subject in need thereof a therapeutically effective amount of a pharmaceutical composition of claim 1.

17. The method of claim 16, wherein the disease or disorder associated with epithelial barrier integrity and/or iNKT cell-mediated inflammatory responses is an inflammatory bowel disease.

18. The method of claim 17, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

19. An assay for detecting the presence of a thiazole/oxazole-modified microcin (TOMM) in a biological sample comprising measuring a level of a TOMM in a biological sample obtained from a subject, wherein if the level of a TOMM is increased at least 1.5 fold relative to a control sample, the biological sample is identified as containing a TOMM.

20. (canceled)

21. (canceled)

22. (canceled)

23. (canceled)

24. The assay of claim 19, wherein if the biological sample is identified as containing a TOMM, the assay further comprises the step of administering a pharmaceutical composition of claim 1.
